(19) United States
(12) Patent Application Publication
(10) Pub. No.: US 2026/0126564 A1
(43) Pub. Date: May 7, 2026

(54) CORE-SHELL COMPOUND, PHOTOSENSITIVE RESIN COMPOSITION INCLUDING THE SAME, PHOTOSENSITIVE RESIN LAYER, COLOR FILTER AND CMOS IMAGE SENSOR

(71) Applicant: SAMSUNG SDI CO., LTD., Yongin-si (KR)

(72) Inventors: Heejo MOON, Suwon-si (KR); Baek Soung PARK, Suwon-si (KR); Kwangwon SEO, Suwon-si (KR); Ickjin KIM, Suwon-si (KR); Subin PARK, Suwon-si (KR)

(21) Appl. No.: 19/230,681

(22) Filed: Jun. 6, 2025

(30) Foreign Application Priority Data
Nov. 1, 2024 (KR) 10-2024-0153668

(51) Int. Cl. G02B 1/04; C07D 401/12; C09B 57/00; C09B 67/02; C09B 67/08; G02B 5/20; G03F 7/00; G03F 7/027; G03F 7/105; H10F 39/00

(52) U.S. Cl. CPC G02B 1/04; C07D 401/12; C09B 57/007; C09B 67/0008; C09B 67/0097; G02B 5/206; G03F 7/0007; G03F 7/027; G03F 7/105; H10F 39/8053

(57) ABSTRACT

A core-shell compound, a photosensitive resin composition including the same, a photosensitive resin layer manufactured using the photosensitive resin composition, a color filter including the photosensitive resin layer, and a CMOS image sensor including the color filter, the core-shell compound including a core represented by Chemical Formula 1 and a shell surrounding the core, the shell being represented by Chemical Formula 2:

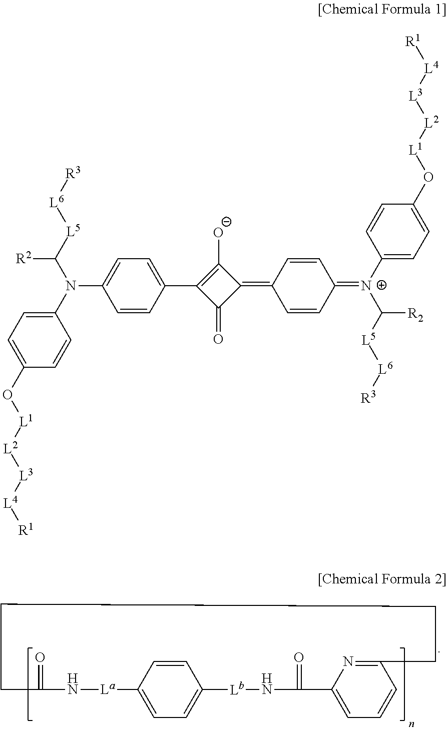

### CROSS-REFERENCE TO RELATED APPLICATION

This application claims priority to and the benefit of Korean Patent Application No. 10-2024-0153668 filed at the Korean Intellectual Property Office on Nov. 1, 2024, the entire contents of which are incorporated herein by reference.

### BACKGROUND

### 1. Field

Embodiments relate to a core-shell compound, a photosensitive resin composition including the same, a photosensitive resin layer manufactured using the photosensitive resin composition, a color filter including the photosensitive resin layer, and a CMOS image sensor including the color filter.

### 2. Description of the Related Art

With the recent rapid development of advanced information and communication processing technology and overall electronics industry, a next generation detector rapidly transmitting and receiving a large amount of information and the development of a new concept device and system have been considered. With the rise of video processing and the like in mobile devices, technology development of an ultra-down-sized and ultra-power-saving image sensor is rapidly accelerated, centering on existing CCD (Charge Coupled Device) and CMOS (Complementary Metal Oxide Semiconductor).

An image sensor, which is a semiconductor that converts photons into electrons and displays them on a display or stores them in a storage device, may include a light receiving element that converts light signals into electrical signals, a pixel circuit portion that amplifies and compresses the converted electrical signals, and an ASIC portion that converts these pre-treated analog signals into digital signals to treat image signals and for example, includes CCD, CMOS, CIS (Contact Image Sensor), and the like.

The CCD and CMOS image sensors use the same light-receiving element, but in the CCD image sensor, charges generated in the light-receiving element sequentially move through MOS capacitors connected in series and are converted into voltages in a source follower connected at the final end. In the CMOS image sensor, the charges are converted into voltages in a source follower built into each pixel and output to the outside. The CCD image sensor moves electrons generated by light as they are to an output unit by using a gate pulse, and the CMOS image sensor convert the electrons generated by light into voltages in each pixel and then output them through several CMOS switches. These image sensors are very widely applied from household products such as a digital camera and a mobile phone to an endoscope used in hospitals and a telescope used in a satellite orbiting the earth.

### SUMMARY

The embodiments may be realized by providing a core-shell compound, including a core represented by Chemical Formula 1 and a shell surrounding the core, the shell being represented by Chemical Formula 2:

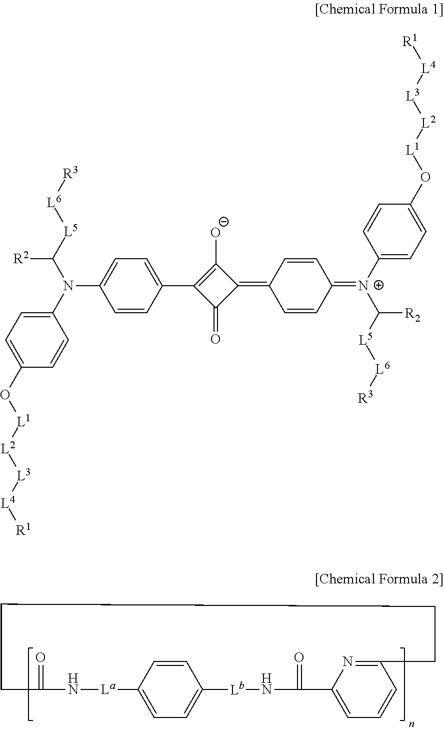

wherein, in Chemical Formula 1 and Chemical Formula 2, R¹ is a thermosetting group, R² and R³ are each independently a hydrogen atom, a substituted or unsubstituted C1 to C20 alkyl group, or a substituted or unsubstituted C1 to C20 alkoxy group, L¹ and L⁶ are each independently a substituted or unsubstituted C1 to C10 alkylene group or a group represented by Chemical Formula L, provided that at least one of L¹ and L⁶ is a group represented by Chemical Formula L, L², L⁴, L^{a}, and L^{b} are each independently a single bond or a substituted or unsubstituted C1 to C10 alkylene group, L³ is a single bond or an ester group represented by *—C(═O)O—* or *—OC(═O)—*, L⁵ is a single bond or a substituted or unsubstituted C1 to C10 alkylene group, and n is an integer greater than or equal to 2,

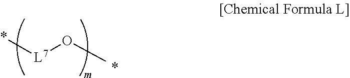

wherein, in Chemical Formula L, L⁷ is a substituted or unsubstituted C1 to C10 alkylene group, and m is an integer of 2 to 10.

The thermosetting group may include a substituted or unsubstituted epoxy group, a substituted or unsubstituted oxetane group, or a combination thereof.

R¹ may be a thermosetting group, R² and R³ may each independently be a hydrogen atom or a substituted or unsubstituted C1 to C20 alkyl group, L¹ and L⁵ may each independently be a substituted or unsubstituted C1 to C10 alkylene group, L² to L⁴ may each independently be a single bond, and L⁶ may be a group represented by Chemical Formula L.

R¹ may be a thermosetting group, R² and R³ may each independently be a hydrogen atom or a substituted or unsubstituted C1 to C20 alkyl group, L¹ may be a group represented by Chemical Formula L, L², L⁴, and L⁶ may each independently be a substituted or unsubstituted C1 to C10 alkylene group, L³ may be an ester group represented by *—C(═O)O—* or *—OC(═O)—*, and L⁵ may be a single bond or a substituted or unsubstituted C1 to C10 alkylene group.

R¹ may be a thermosetting group, R² and R³ may each independently be a hydrogen atom or a substituted or unsubstituted C1 to C20 alkyl group, L¹ and L⁶ may each independently be a group represented by Chemical Formula L, L² and L⁴ may each independently be a substituted or unsubstituted C1 to C10 alkylene group, L³ may be an ester group represented by *—C(═O)O—* or *—OC(═O)—*, and L⁵ may be a substituted or unsubstituted C1 to C10 alkylene group.

The core represented by Chemical Formula 1 may have a maximum absorption wavelength at about 610 nm to about 640 nm.

The shell represented by Chemical Formula 2 may be represented by Chemical Formula 2-1:

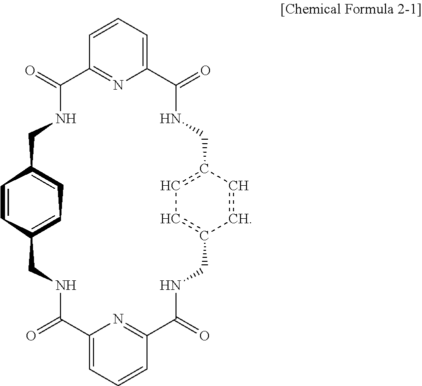

The core-shell compound may be represented by one of Chemical Formula A to Chemical Formula C:

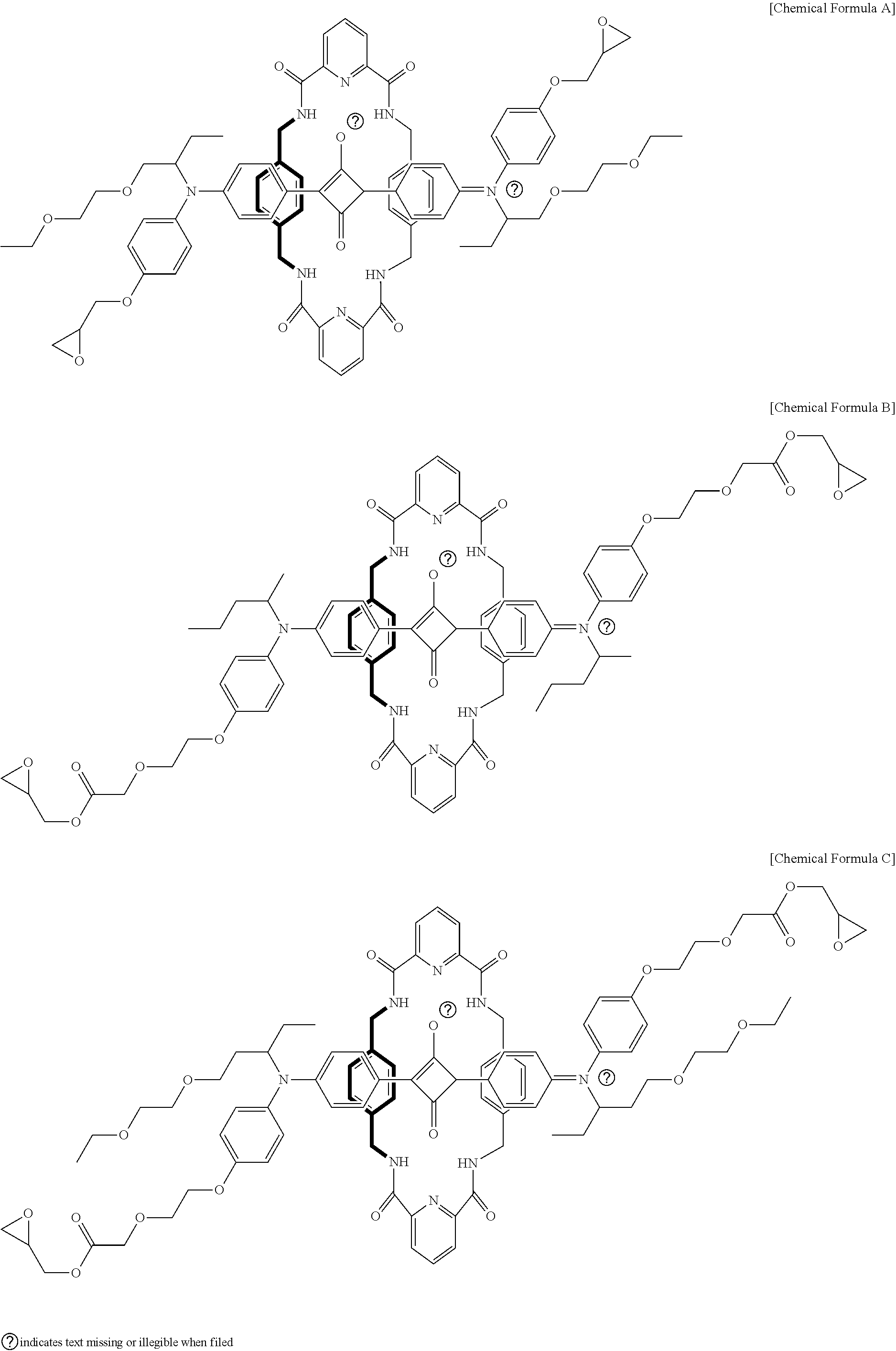

The core-shell compound may be a green dye.

The embodiments may be realized by providing a photosensitive resin composition including the core-shell compound according to an embodiment.

The photosensitive resin composition may further include a binder resin, a photopolymerizable monomer, a photopolymerization initiator, a pigment, or a solvent.

The photosensitive resin composition may be used for a CMOS image sensor.

The embodiments may be realized by providing a photosensitive resin layer manufactured using the photosensitive resin composition according to an embodiment.

The embodiments may be realized by providing a color filter including the photosensitive resin layer according to an embodiment.

The embodiments may be realized by providing a CMOS image sensor including the color filter according to an embodiment.

### DETAILED DESCRIPTION

Example embodiments will now be described more fully hereinafter; however, they may be embodied in different forms and should not be construed as limited to the embodiments set forth herein. Rather, these embodiments are provided so that this disclosure will be thorough and complete, and will fully convey exemplary implementations to those skilled in the art. As used herein, the term “or” is not an exclusive term, e.g., “A or B” would include A, B, or A and B.

As used herein, when specific definition is not otherwise provided, “substituted” refers to one substituted with a substituent selected from a halogen (F, Br, Cl, or I), a hydroxy group, a nitro group, a cyano group, an amino group (NH₂, NH(R²⁰⁰), or N(R²⁰¹)(R²⁰²), wherein R²⁰⁰, R²⁰¹, and R²⁰² are the same or different, and are each independently a C1 to C10 alkyl group), an amidino group, a hydrazine group, a hydrazone group, a carboxyl group, a substituted or unsubstituted alkyl group, a substituted or unsubstituted alkenyl group, a substituted or unsubstituted alkynyl group, a substituted or unsubstituted alicyclic organic group, a substituted or unsubstituted aryl group, and a substituted or unsubstituted heterocyclic group.

As used herein, when specific definition is not otherwise provided, “alkyl group” refers to a C1 to C20 alkyl group, and specifically a C1 to C15 alkyl group, “cycloalkyl group” refers to a C3 to C20 cycloalkyl group, and specifically a C3 to C18 cycloalkyl group, “alkoxy group” refers to a C1 to C20 alkoxy group, and specifically a C1 to C18 alkoxy group, “aryl group” refers to a C6 to C20 aryl group, and specifically a C6 to C18 aryl group, “alkenyl group” refers to a C2 to C20 alkenyl group, and specifically a C2 to C18 alkenyl group, “alkylene group” refers to a C1 to C20 alkylene group, and specifically C1 to C18 alkylene group, and “arylene group” refers to a C6 to C20 arylene group, and specifically a C6 to C₁₆ arylene group.

As used herein, when specific definition is not otherwise provided, “(meth)acrylate” refers to “acrylate” and “methacrylate” and “(meth)acrylic acid” refers to “acrylic acid” and “methacrylic acid.”

As used herein, when specific definition is not otherwise provided, “combination” refers to mixing or copolymerization. In addition, “copolymerization” refers to block copolymerization or random copolymerization, and “copolymer” refers to block copolymerization or random copolymerization.

In the chemical formula of the present specification, unless a specific definition is otherwise provided, hydrogen is boned at the position when a chemical bond is not drawn where supposed to be given.

In the present specification, when specific definition is not otherwise provided, “*” indicates a point where the same or different atom or chemical formula is linked.

Some example embodiments provide a core-shell compound including a core represented by Chemical Formula 1 and a shell surrounding the core and represented by Chemical Formula 2.

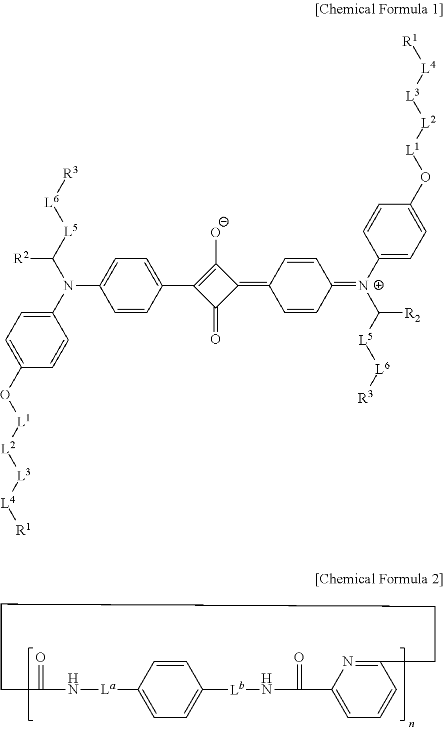

In Chemical Formula 1 and Chemical Formula 2, R₁ may be, e.g., a thermosetting group.

R² and R³ may each independently be or include, e.g., a hydrogen atom, a substituted or unsubstituted C1 to C20 alkyl group, or a substituted or unsubstituted C1 to C20 alkoxy group.

L¹ and L⁶ may each independently be or include, e.g., a substituted or unsubstituted C1 to C10 alkylene group or a group represented by Chemical Formula L, provided that at least one of L¹ and L⁶ is represented by Chemical Formula L.

L², L⁴, L^{a}, and L^{b} may each independently be or include, e.g., a single bond or a substituted or unsubstituted C1 to C10 alkylene group.

L³ may be or include, e.g., a single bond or an ester group, e.g., *—C(═O)O—* or *—OC(═O)—*.

L⁵ may be or include, e.g., a single bond or a substituted or unsubstituted C₁ to C10 alkylene group.

n may be, e.g., an integer greater than or equal to 2.

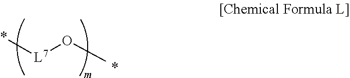

In Chemical Formula L, L⁷ may be or include, e.g., a substituted or unsubstituted C1 to C10 alkylene group.

m may be, e.g., an integer of 1 to 10, e.g., an integer of 2 to 10.

A liquid crystal display device among many types of displays may have an advantage of lightness, thinness, low cost, low power consumption for operation, and improved adherence to an integrated circuit and has been more widely used for a laptop computer, a monitor, and a TV screen. The liquid crystal display device may include a lower substrate on which a black matrix, a color filter, and an ITO pixel electrode may be formed, and an upper substrate on which an active circuit portion including a liquid crystal layer, a thin film transistor, and a capacitor layer and an ITO pixel electrode may be formed. Color filters may be formed in a pixel region by sequentially stacking a plurality of color filters (e.g., formed of three primary colors such as red (R), green (G), and blue (B)) in a predetermined order to form each pixel, and a black matrix layer may be disposed in a predetermined pattern on a transparent substrate to form a boundary between the pixels. The pigment dispersion method, e.g., a method of forming a color filter, may provide a colored thin film by repeating a series of processes such as coating a photopolymerizable composition including a colorant on a transparent substrate including a black matrix, exposing a formed pattern to light, removing a non-exposed part with a solvent, and thermally curing the same. A coloring photosensitive resin composition used for manufacturing a color filter according to the pigment dispersion method may include an alkali soluble resin, a photopolymerization monomer, a photopolymerization initiator, an epoxy resin, a solvent, other additives, or the like. The pigment dispersion method may be used to manufacture LCDs used in, e.g., mobile phones, laptops, monitors, and TVs.

An image sensor may be a part used for photographing images in a portable phone camera or DSC (a digital still camera). It may be classified as, e.g., a charge-coupled device (CCD) image sensor or a complementary metal oxide semiconductor (CMOS) image sensor depending upon the manufacturing process and the application method. A color imaging device for a charge-coupled device image sensor or a complementary metal oxide semiconductor image sensor may include color filters each having filter segments of mixing primary color of red, green, and blue, and the colors may be separated. Some color filters mounted in color imaging devices may have a pattern size of 2 μm or less, which may be 1/100th to 1/200th of the pattern size of other color filter patterns for LCDs. Accordingly, increased resolution and decreased pattern residues may be important factors for determining the performance of a device.

Meanwhile, a color filter formed of a pigment photosensitive resin composition may have a color mixing problem due to particle sizes of the pigment and a limit to film-thinning. In addition, a color imaging device for an image sensor may use a smaller dispersed particle size to form a fine pattern. Accordingly, efforts to improve resolution by introducing a non-particle dye instead of the pigment to prepare a photosensitive resin composition suitable for the dye have been considered.

The embodiments may relate to a green dye for use in a color filter for a CMOS image sensor or a color filter for OLEDos (OLED on silicon). As pixels have a smaller size, there may be a limit to manufacture a fine pattern by using a pigment, and accordingly, a dye may be used to compensate for this. A dye may have issues in terms of processability during the pattern manufacturing, e.g., it may exhibit much deteriorated developability, it may be very difficult to form a fine pattern after the curing and thermal process. In addition, when the dye as a colorant is included in a small amount based on a total amount of the composition, the developability of the dye itself may be a little insufficient. When the photosensitive resin composition for a CMOS image sensor or OLEDos (OLED on silicon) includes the dye in an excessive amount (about 15 wt % to about 30 wt %, e.g., about 16 wt % to about 27 wt %, based on the total weight of the photosensitive resin composition), a dye with excellent developability itself may be used.

Embodiments relate to a core-shell compound synthesized by inserting an alkylene oxide linking group into a squarylium compound forming a core and introducing a thermosetting group at the terminal end thereof, and surrounding the core with a shell. The core-shell compound according to some example embodiments synthesized in this manner may have excellent developability on its own, and a photosensitive resin composition including the same in excess as a green dye may not exhibit a significant decrease in developability even after going through a curing and heat process, and may therefore be highly suitable for use in a green color filter for a CMOS image sensor or an OLEDos (OLED on silicon).

That is, some embodiments may provide a squarylium core-shell dye with improved developability. As described above, there may be an issue of occurrence of bottom residue when producing a color filter using a composition including a dye, and the cause of this may be low solubility of the dye in an alkaline aqueous solution used in the developing process. Though use of some embodiments, bottom residue may be reduced by including an alkylene oxide linking group, such as ethylene oxide, which may help increase water solubility, in the dye structure while introducing a thermosetting group at the terminal end.

In an implementation, the thermosetting group may include, e.g., a substituted or unsubstituted epoxy group, a substituted or unsubstituted oxetane group, or a combination thereof.

The thermosetting group may be further substituted with, e.g., an alkyl group or the like.

In an implementation, the compound represented by Chemical Formula 1 may be, e.g., 1) a compound represented by Chemical Formula 1, wherein R¹ may be, e.g., a thermosetting group, R² and R³ may each independently be, e.g., a hydrogen atom or a substituted or unsubstituted C1 to C20 alkyl group, L¹ and L⁵ may each independently be, e.g., a substituted or unsubstituted C1 to C10 alkylene group, L² to L⁴ may each independently be, e.g., a single bond, and L⁶ may be, e.g., a group represented by Chemical Formula L, 2) a compound represented by Chemical Formula 1, wherein R¹ may be, e.g., a thermosetting group, R² and R³ may each independently be, e.g., a hydrogen atom or a substituted or unsubstituted C1 to C20 alkyl group, L¹ may be, e.g., a group represented by Chemical Formula L, L², L⁴, and L⁶ may each independently be, e.g., a substituted or unsubstituted C1 to C10 alkylene group, L³ may be an ester group, e.g., *—C(═O)O—* or *—OC(═O)—*, and L⁵ may be, e.g., a single bond or a substituted or unsubstituted C1 to C10 alkylene group, or 3) a compound represented by Chemical Formula 1, wherein R¹ may be, e.g., a thermosetting group, R² and R³ may each independently be, e.g., a hydrogen atom or a substituted or unsubstituted C1 to C20 alkyl group, L¹ and L⁶ may each independently be, e.g., a group represented by Chemical Formula L, L² and L⁴ may each independently be, e.g., a substituted or unsubstituted C₁ to C10 alkylene group, L³ may be an ester group, e.g., *—C(═O)O—* or *—OC(═O)—*, and L⁵ may be, e.g., a substituted or unsubstituted C1 to C10 alkylene group, and all of the three types of compounds may provide a dye with improved developability.

In an implementation, the core represented by Chemical Formula 1 may include, e.g., a total of 2, 4, or 6 thermosetting groups at the terminal ends. The core represented by Chemical Formula 1 having a total of 1, 3, or 5 terminal thermosetting groups may be, e.g., difficult to synthesize due to its structure and if it is synthesized, it may be expensive to apply to an actual line.

In an implementation, the core represented by Chemical Formula 1 may have better developability when the number of terminal thermosetting groups is 6 than when the number of terminal thermosetting groups is 4, and may have better developability when the number of terminal thermosetting groups is 4 than when the number of terminal thermosetting groups is 2. That is, if only the developability aspect of the compound is considered, the number of terminal thermosetting groups in the core structure represented by Chemical Formula 1 may be better to be 4 than 2, and may be better to be 6 than 4.

In addition, the core represented by Chemical Formula 1 may be advantageous in terms of solubility as the number of alkylene oxide linkages increases, and the alkylene oxide linkages may be, e.g., an ethylene oxide linkage or a propylene oxide linkage. Among these, the ethylene oxide linkage is a more hydrophilic linkage, so using the ethylene oxide linkage may be more advantageous in terms of solubility.

In an implementation, in the core represented by Chemical Formula 1, when the number of the terminal thermosetting groups is 1, not only may synthesis be difficult as described above, but the developability may be lower than when the number of the terminal thermosetting groups is 2. Therefore, it may be desirable that the core represented by Chemical Formula 1 may include two or more terminal thermosetting groups.

In an implementation, the core represented by Chemical Formula 1 may have a maximum absorption wavelength at about 610 nm to about 640 nm. Even if a dye compound has excellent solubility in an organic solvent of 10% or more, if it does not have a maximum absorption wavelength of about 610 nm to about 640 nm, it may be unsuitable for use as a green photosensitive resin composition for a CMOS image sensor or OLEDos (OLED on silicon) due to low transmittance.

The shell represented by Chemical Formula 2 may be represented by Chemical Formula 2-1.

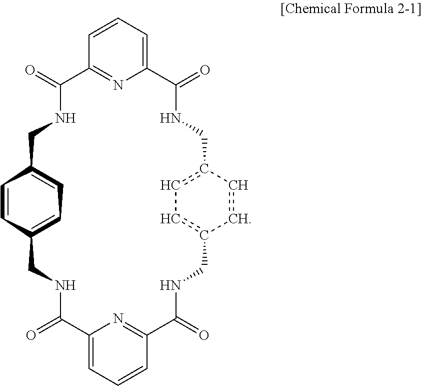

In an implementation, the core-shell compound may be represented by one of Chemical Formula A to Chemical Formula C.

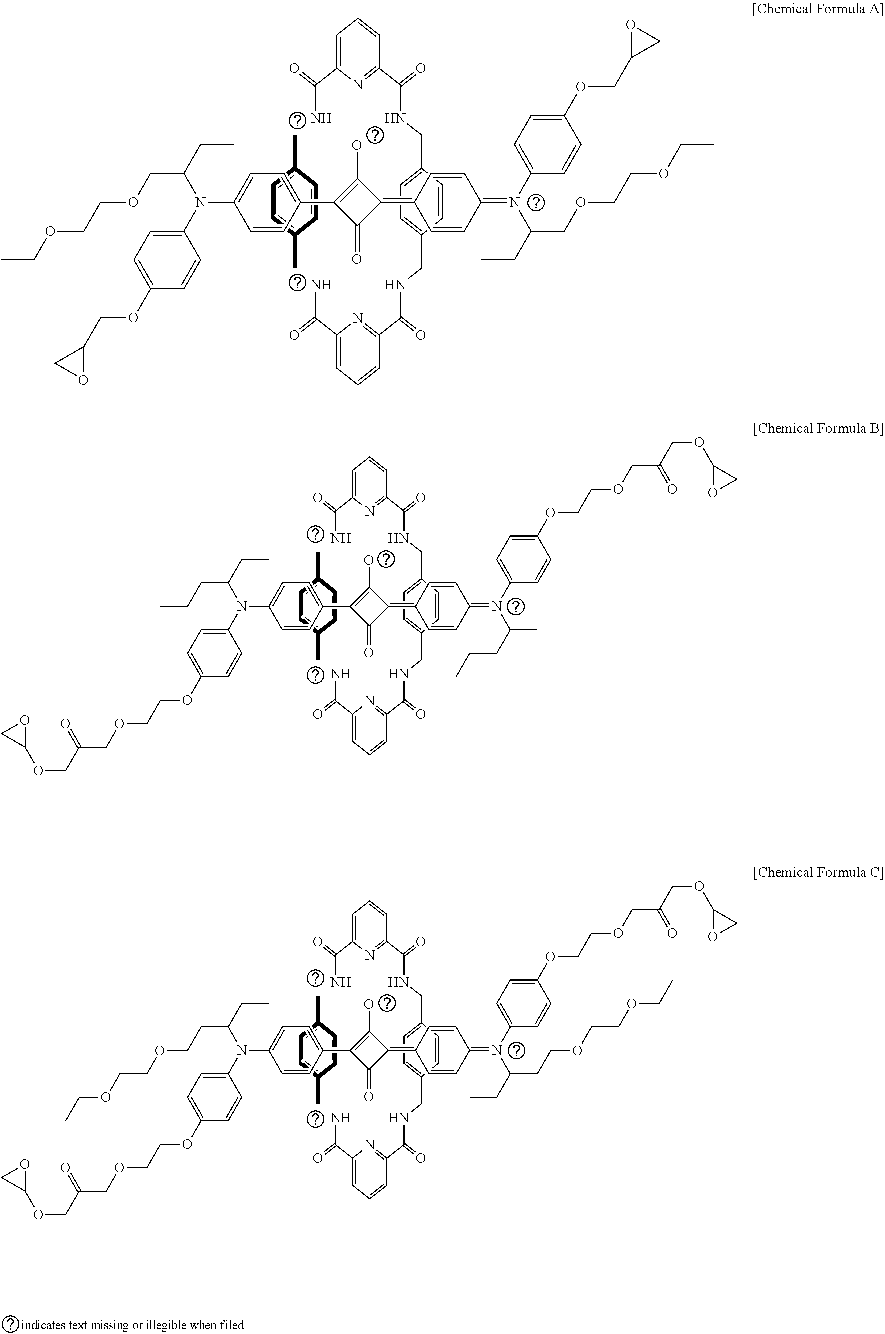

In an implementation, the core-shell compound may be, e.g., a green dye.

According to some example embodiments, a photosensitive resin composition including the core-shell compound according to the example embodiments may be provided.

In an implementation, the photosensitive resin composition may have a transmittance of, e.g., about 90% or more at about 540 nm, a transmittance of, e.g., about 10% or less at about 600 nm to about 640 nm, and a transmittance of, e.g., about 5% or less at about 450 nm, and may be suitable for implementing a green color filter for a high-transmittance CIS. In an implementation, the photosensitive resin composition may be used for a high-transmittance CMOS image sensor or OLEDos (OLED on silicon).

The photosensitive resin composition may include, e.g., the core-shell compound, and may further include a binder resin, a photopolymerizable monomer, a photopolymerization initiator, a pigment, or a solvent.

According to some example embodiments, the core-shell compound may act as a dye and as a colorant in a photosensitive resin composition. When the colorant is used in a hybrid form of the core-shell dye and the pigment, the developability may be maximized, rather than when the core-shell dye is used alone. That is, the core-shell compound according to some example embodiments may be a hybrid type dye used together with a pigment.

In an implementation, the pigment may further include, e.g., a yellow pigment, a green pigment, or a combination thereof.

The yellow pigment may be, e.g., C.I. pigment yellow 138, C.I. pigment yellow 139, C.I. pigment yellow 150, or the like, in a color index, and these may be used alone or as a mixture of two or more.

The green pigment may be, e.g., C.I. pigment green 36, C.I. pigment green 58, C.I. pigment green 59, or the like, in a color index, and these may be used alone or as a mixture of two or more.

The pigment may be included in the photosensitive resin composition in the form of pigment dispersion.

The pigment dispersion may include, e.g., a solid pigment, a solvent, or a dispersant for uniformly dispersing the pigment in the solvent.

The solid pigment may be included in an amount of about 1 wt % to about 20 wt %, e.g., about 8 wt % to about 20 wt %, about 8 wt % to about 15 wt %, about 10 wt % to about 20 wt %, or about 10 wt % to about 15 wt %, based on a total weight of pigment dispersion.

The dispersant may be a non-ionic dispersant, an anionic dispersant, a cationic dispersant, or the like. In an implementation, the dispersant may be polyalkylene glycol and esters thereof, polyoxyalkylene, a polyhydric alcohol ester alkylene oxide addition product, an alcohol alkylene oxide addition product, sulfonate ester, a sulfonate salt, a carboxylate ester, a carboxylate salt, an alkylamide alkylene oxide addition product, alkyl amine, or the like, and these may be used alone or as a mixture of two or more.

Commercially available examples of the dispersant may include, e.g., DISPERBYK-101, DISPERBYK-130, DISPERBYK-140, DISPERBYK-160, DISPERBYK-161, DISPERBYK-162, DISPERBYK-163, DISPERBYK-164, DISPERBYK-165, DISPERBYK-166, DISPERBYK-170, DISPERBYK-171, DISPERBYK-182, DISPERBYK-2000, DISPERBYK-2001, or the like made by BYK Co., Ltd.; EFKA-47, EFKA-47EA, EFKA-48, EFKA-49, EFKA-100, EFKA-400, EFKA-450, or the like made by EFKA Chemicals Co.; Solsperse 5000, Solsperse 12000, Solsperse 13240, Solsperse 13940, Solsperse 17000, Solsperse 20000, Solsperse24000GR, Solsperse 27000, Solsperse 28000, or the like made by Zeneka Co.; or PB711, PB821, or the like made by Ajinomoto Inc.

The dispersant may be included in an amount of, e.g., about 1 wt % to about 20 wt %, based on the total weight of the pigment dispersion. If the dispersant is included within this range, dispersion of a photosensitive resin composition may be improved due to an appropriate viscosity, and thus optical, physical and chemical quality may be maintained when the photosensitive resin composition is applied to products.

A solvent for forming the pigment dispersion may be, e.g., ethylene glycol acetate, ethylcellosolve, propylene glycol methyletheracetate, ethyllactate, polyethylene glycol, cyclohexanone, propylene glycol methylether, or the like.

The pigment dispersion may be included in an amount of about 10 wt % to about 20 wt %, e.g., about 12 wt % to about 18 wt %, based on the total weight of the photosensitive resin composition. If the pigment dispersion is included within the above range, it may be advantageous to secure a process margin, and may have improved color gamut and contrast ratio.

The binder resin may be, e.g., an acrylic binder resin.

The acrylic binder resin may be, e.g., a copolymer of a first ethylenic unsaturated monomer and a second ethylenic unsaturated monomer that is copolymerizable therewith, and may be, e.g., a resin including at least one acrylic repeating unit.

The first ethylenic unsaturated monomer may be, e.g., an ethylenic unsaturated monomer including at least one carboxyl group and examples of the monomer may include, e.g., acrylic acid, methacrylic acid, maleic acid, itaconic acid, fumaric acid, or a combination thereof.

The first ethylenic unsaturated monomer may be included in an amount of about 5 wt % to about 50 wt %, e.g., about 10 wt % to about 40 wt %, based on the total weight of the acrylic binder resin.

The second ethylenic unsaturated monomer may be, e.g., an aromatic vinyl compound such as styrene, α-methylstyrene, vinyl toluene, vinylbenzylmethylether, or the like; an unsaturated carboxylate ester compound such as methyl(meth)acrylate, ethyl(meth)acrylate, butyl(meth)acrylate, 2-hydroxyethyl(meth)acrylate, 2-hydroxy butyl(meth)acrylate, benzyl(meth)acrylate, cyclohexyl(meth)acrylate, phenyl(meth)acrylate, or the like; an unsaturated amino alkyl carboxylate ester compound such as 2-aminoethyl(meth)acrylate, 2-dimethylaminoethyl(meth)acrylate, or the like; a carboxylic acid vinyl ester compound such as vinyl acetate, vinyl benzoate, or the like; an unsaturated glycidyl carboxylate ester compound such as glycidyl(meth)acrylate, or the like; a vinyl cyanide compound such as (meth)acrylonitrile or the like; an unsaturated amide compound such as (meth)acrylamide, or the like; or the like, and may be used alone or as a mixture of two or more.

In an implementation, the acrylic binder resin may be, e.g., a (meth)acrylic acid/benzylmethacrylate copolymer, a (meth)acrylic acid/benzylmethacrylate copolymer, a (meth)acrylic acid/benzylmethacrylate/styrene copolymer, a (meth)acrylic acid/benzylmethacrylate/2-hydroxyethylmethacrylate copolymer, a (meth)acrylic acid/benzylmethacrylate/styrene/2-hydroxyethylmethacrylate copolymer, or the like, and these may be used alone or as a mixture of two or more.

A weight average molecular weight of the binder resin may be about 3,000 g/mol to about 150,000 g/mol, e.g., about 5,000 g/mol to about 50,000 g/mol or about 20,000 g/mol to about 30,000 g/mol. If the binder resin has a weight average molecular weight within these ranges, the photosensitive resin composition may have good physical and chemical properties, appropriate viscosity, and close-contacting properties with a substrate during manufacture of a color filter.

An acid value of the binder resin may be about 15 mgKOH/g to about 60 mgKOH/g, e.g., about 20 mgKOH/g to about 50 mgKOH/g. When the acid value of the binder resin is within these ranges, resolution of pixel patterns may be improved.

The binder resin may be included in an amount of about 1 wt % to about 30 wt %, e.g., about 1 wt % to about 20 wt %, based on the total weight of the photosensitive resin composition. If the binder resin is included within these ranges, the composition may have an excellent developability and improved crosslinking, and thus may have excellent surface flatness when manufactured into a color filter.

The photopolymerizable monomer may be, e.g., a mono-functional or multi-functional ester of (meth)acrylic acid including at least one ethylenic unsaturated double bond.

The photopolymerizable monomer may have an ethylenic unsaturated double bond and thus, may have sufficient polymerization during exposure in a pattern-forming process and form a pattern having excellent heat resistance, light resistance, and chemical resistance.

Examples of the photopolymerizable monomer may include ethylene glycol di(meth)acrylate, diethylene glycol di(meth)acrylate, triethylene glycol di(meth)acrylate, propylene glycol di(meth)acrylate, neopentyl glycol di(meth)acrylate, 1,4-butanediol di(meth)acrylate, 1,6-hexanediol di(meth)acrylate, bisphenol A di(meth)acrylate, pentaerythritol di(meth)acrylate, pentaerythritol tri(meth)acrylate, pentaerythritol tetra(meth)acrylate, pentaerythritol hexa(meth)acrylate, dipentaerythritol di(meth)acrylate, dipentaerythritol tri(meth)acrylate, dipentaerythritol penta(meth)acrylate, dipentaerythritol hexa(meth)acrylate, bisphenol A epoxy(meth)acrylate, ethylene glycol monomethylether (meth)acrylate, trimethylol propane tri(meth)acrylate, tris(meth)acryloyloxyethyl phosphate, novolac epoxy (meth)acrylate, or the like.

Commercially available examples of the photopolymerizable monomer are as follows. The mono-functional (meth)acrylic acid ester may include, e.g., Aronix M-101®, M-111®, M-114® (Toagosei Chemistry Industry Co., Ltd.); KAYARAD TC-110S®, TC-120S® (Nippon Kayaku Co., Ltd.); V-158®, V-2311® (Osaka Organic Chemical Ind., Ltd.), and the like. Examples of a difunctional (meth)acrylic acid ester may include, e.g., Aronix M-210®, M-240®, M-6200® (Toagosei Chemistry Industry Co., Ltd.), KAYARAD HDDA®, HX-220®, R-604® (Nippon Kayaku Co., Ltd.), V-260®, V-312®, V-335 HP® (Osaka Organic Chemical Ind., Ltd.), or the like. Examples of a tri-functional (meth)acrylic acid ester may include, e.g., Aronix M-309®, M-400®, M-405®, M-450®, M-710®, M-8030®, M-8060®, or the like of Toagosei Chemistry Industry Co., Ltd.; KAYARAD TMPTA®, DPCA-20®, DPCA-30®, DPCA-60®, DPCA-120® of Nippon Kayaku Co., Ltd., V-295®, V-300®, V-360®, V-GPT®, V-3PA®, V-400® of Osaka Yuki Kayaku Kogyo Co. Ltd., or the like. These may be used alone or as a mixture of two or more.

The photopolymerizable monomer may be treated with acid anhydride to improve developability.

The photopolymerizable monomer may be included in an amount of about 1 wt % to about 15 wt %, e.g., about 5 wt % to about 10 wt %, based on the total weight of the photosensitive resin composition. If the photopolymerizable monomer is included within the ranges, the photopolymerizable monomer may be sufficiently cured during exposure in a pattern-forming process and may have excellent reliability, and developability for alkali developing solution may be improved.

The photopolymerization initiator may be a generally-used initiator in a photosensitive resin composition, e.g., an acetophenone compound, a benzophenone compound, a thioxanthone compound, a benzoin compound, a triazine compound, an oxime compound, or a combination thereof.

Examples of the acetophenone compound may be 2,2′-diethoxy acetophenone, 2,2′-dibutoxy acetophenone, 2-hydroxy-2-methylpropinophenone, p-t-butyltrichloro acetophenone, p-t-butyldichloro acetophenone, 4-chloro acetophenone, 2,2′-dichloro-4-phenoxy acetophenone, 2-methyl-1-(4-(methylthio)phenyl)-2-morpholinopropan-1-one, 2-benzyl-2-dimethylamino-1-(4-morpholinophenyl)-butan-1-one, or the like.

Examples of the benzophenone compound may be benzophenone, benzoyl benzoate, benzoyl methyl benzoate, 4-phenyl benzophenone, hydroxy benzophenone, acrylated benzophenone, 4,4′-bis(dimethylamino)benzophenone, 4,4′-bis(diethylamino)benzophenone, 4,4′-dimethylaminobenzophenone, 4,4′-dichlorobenzophenone, 3,3′-dimethyl-2-methoxybenzophenone, or the like.

Examples of the thioxanthone compound may be thioxanthone, 2-methylthioxanthone, isopropyl thioxanthone, 2,4-diethyl thioxanthone, 2,4-diisopropyl thioxanthone, 2-chlorothioxanthone, or the like.

Examples of the benzoin compound may be benzoin, benzoin methyl ether, benzoin ethyl ether, benzoin isopropyl ether, benzoin isobutyl ether, benzyldimethylketal, or the like.

Examples of the triazine compound may be 2,4,6-trichloro-s-triazine, 2-phenyl 4,6-bis(trichloromethyl)-s-triazine, 2-(3′,4′-dimethoxystyryl)-4,6-bis(trichloromethyl)-s-triazine, 2-(4′-methoxynaphthyl)-4,6-bis(trichloromethyl)-s-triazine, 2-(p-methoxyphenyl)-4,6-bis(trichloromethyl)-s-triazine, 2-(p-tolyl)-4,6-bis(trichloromethyl)-s-triazine, 2-biphenyl 4,6-bis(trichloromethyl)-s-triazine, bis(trichloromethyl)-6-styryl-s-triazine, 2-(naphthol-yl)-4,6-bis(trichloromethyl)-s-triazine, 2-(4-methoxynaphthol-yl)-4,6-bis(trichloromethyl)-s-triazine, 2-4-bis(trichloromethyl)-6-piperonyl-s-triazine, 2-4-bis(trichloromethyl)-6-(4-methoxystyryl)-s-triazine, or the like.

Examples of the oxime compound may include an O-acyloxime compound, 2-(o-benzoyloxime)-1-[4-(phenylthio)phenyl]-1,2-octandione, 1-(o-acetyloxime)-1-[9-ethyl-6-(2-methylbenzoyl)-9H-carbazol-3-yl]ethanone, O-ethoxycarbonyl-a-oxyamino-1-phenylpropan-1-one or the like. Specific examples of the O-acyloxime compound may be 1,2-octandione, 2-dimethylamino-2-(4-methylbenzyl)-1-(4-morpholin-4-yl-phenyl)-butan-1-one, 1-(4-phenylsulfanyl phenyl)-butane-1,2-dione2-oxime-O-benzoate, 1-(4-phenylsulfanyl phenyl)-octane-1,2-dione2-oxime-O-benzoate, 1-(4-phenylsulfanyl phenyl)-octan-1-oneoxime-O-acetate, 1-(4-phenylsulfanyl phenyl)-butan-1-oneoxime-O-acetate, or the like.

The photopolymerization initiator may include, e.g., a carbazole compound, a diketone compound, a sulfonium borate compound, a diazo compound, an imidazole compound, a biimidazole compound, a fluorene compound, or the like, besides the compounds.

The photopolymerization initiator may be used with a photosensitizer capable of causing a chemical reaction by absorbing light and becoming excited and then, transferring its energy.

Examples of the photosensitizer may include tetraethylene glycol bis-3-mercapto propionate, pentaerythritol tetrakis-3-mercapto propionate, dipentaerythritol tetrakis-3-mercapto propionate, or the like.

The photopolymerization initiator may be included in an amount of about 0.01 wt % to about 10 wt %, e.g., about 0.1 wt % to about 5 wt %, based on the total weight of the photosensitive resin composition. If the photopolymerization initiator is included within these ranges, sufficient photopolymerization may occur during exposure in a pattern-forming process, excellent reliability may be realized, heat resistance, light resistance, and chemical resistance of patterns, resolution and close-contacting properties may be improved, and decrease of transmittance due to a non-reaction initiator may be prevented.

The solvent may be a material having compatibility with the core-shell compound according to some example embodiments, the binder resin, the photopolymerizable monomer, the photopolymerization initiator, and the pigment, but not reacting therewith.

Examples of the solvent may include alcohols such as methanol, ethanol, or the like; ethers such as dichloroethyl ether, n-butyl ether, diisoamyl ether, methylphenyl ether, tetrahydrofuran, or the like; glycol ethers such as ethylene glycol monomethylether, ethylene glycol monoethylether, or the like; cellosolve acetates such as methyl cellosolve acetate, ethyl cellosolve acetate, diethyl cellosolve acetate, or the like; carbitols such as methylethyl carbitol, diethyl carbitol, diethylene glycol monomethylether, diethylene glycol monoethylether, diethylene glycol dimethylether, diethylene glycol methylethylether, diethylene glycol diethylether, or the like; propylene glycol alkylether acetates such as propylene glycol monomethylether acetate, propylene glycol propylether acetate, or the like; aromatic hydrocarbons such as toluene, xylene, r the like; ketones such as methylethylketone, cyclohexanone, 4-hydroxy-4-methyl-2-pentanone, methyl-n-propylketone, methyl-n-butylketone, methyl-n-amylketone, 2-heptanone, or the like; saturated aliphatic monocarboxylic acid alkyl esters such as ethyl acetate, n-butyl acetate, isobutyl acetate, or the like; lactic acid esters such as methyl lactate, ethyl lactate, or the like; oxy acetic acid alkyl esters such as oxy methyl acetate, oxy ethyl acetate, oxy butyl acetate, or the like; alkoxy acetic acid alkyl esters such as methoxy methyl acetate, methoxy ethyl acetate, methoxy butyl acetate, ethoxy methyl acetate, ethoxy ethyl acetate, or the like; 3-oxy propionic acid alkylesters such as 3-oxy methyl propionate, 3-oxy ethyl propionate, or the like; 3-alkoxy propionic acid alkyl esters such as 3-methoxy methyl propionate, 3-methoxy ethyl propionate, 3-ethoxy ethyl propionate, 3-ethoxy methyl propionate, or the like; 2-oxy propionic acid alkyl esters such as 2-oxy methyl propionate, 2-oxy ethyl propionate, 2-oxy propyl propionate, or the like; 2-alkoxy propionic acid alkyl esters such as 2-methoxy methyl propionate, 2-methoxy ethyl propionate, 2-ethoxy ethyl propionate, 2-ethoxy methyl propionate, or the like; 2-oxy-2-methyl propionic acid esters such as 2-oxy-2-methyl methyl propionate, 2-oxy-2-methyl ethyl propionate, or the like, monooxy monocarboxylic acid alkyl esters of 2-alkoxy-2-methyl alkyl propionates such as 2-methoxy-2-methyl methyl propionate, 2-ethoxy-2-methyl ethyl propionate, or the like; esters such as 2-hydroxy ethyl propionate, 2-hydroxy-2-methyl ethyl propionate, hydroxy ethyl acetate, 2-hydroxy-3-methyl methyl butanoate, or the like; ketonate esters such as ethyl pyruvate, or the like, and in addition, a high boiling point solvent such as N-methylformamide, N,N-dimethylformamide, N-methylformanilide, N-methylacetamide, N,N-dimethylacetamide, N-methylpyrrolidone, dimethylsulfoxide, benzylethylether, dihexylether, acetylacetone, isophorone, caproic acid, caprylic acid, 1-octanol, 1-nonanol, benzylalcohol, benzyl acetate, ethyl benzoate, diethyl oxalate, diethyl maleate, γ-butyrolactone, ethylene carbonate, propylene carbonate, phenyl cellosolve acetate, or the like may also be used.

Considering compatibility and reactivity, glycol ethers such as ethylene glycol monoethylether, or the like; ethylene glycol alkylether acetates such as ethyl cellosolve acetate, or the like; esters such as 2-hydroxy ethyl propionate, or the like; carbitols such as diethylene glycol monomethylether, or the like; propylene glycol alkylether acetates such as propylene glycol monomethylether acetate, propylene glycol propylether acetate, or the like; and/or ketones such as cyclohexanone, or the like may be desirably used.

The solvent may be included in a balance amount, e.g., about 30 wt % to about 80 wt %, based on the total weight of the photosensitive resin composition. If the solvent is included within these ranges, the photosensitive resin composition may have an appropriate viscosity resulting in improvement of coating characteristics of a color filter.

The photosensitive resin composition according to some example embodiments may further include an epoxy compound in order to help improve close-contacting properties with a substrate.

Examples of the epoxy compound may include a phenol novolac epoxy compound, a tetramethyl biphenyl epoxy compound, a bisphenol A epoxy compound, an alicyclic epoxy compound, or a combination thereof.

The epoxy compound may be included in an amount of about 0.01 parts by weight to about 20 parts by weight, e.g., about 0.1 parts by weight to about 10 parts by weight based on 100 parts by weight of the photosensitive resin composition. If the epoxy compound is included within the above ranges, close-contacting properties, storage properties, or the like may be improved.

In an implementation, the photosensitive resin composition may further include a silane coupling agent having a reactive substituent, e.g., a carboxyl group, a methacryloyl group, an isocyanate group, an epoxy group, or the like to help improve its adhesion to a substrate.

Examples of the silane coupling agent may include trimethoxysilyl benzoic acid, γ-methacryl oxypropyl trimethoxysilane, vinyl triacetoxysilane, vinyl trimethoxysilane, γ-isocyanate propyl triethoxysilane, γ-glycidoxy propyl trimethoxysilane, β-(epoxycyclohexyl)ethyltrimethoxysilane, or the like and these may be used alone or in a mixture of two or more.

The silane coupling agent may be included in an amount of, e.g., about 0.01 parts by weight to about 10 parts by weight, based on 100 parts by weight of the photosensitive resin composition. If the silane coupling agent is included within the range, close-contacting properties, storing properties, and the like may be improved.

In an implementation, the photosensitive resin composition may further include a surfactant in order to improve coating properties and prevent a defect if necessary.

Examples of the surfactant may be fluorine surfactants that are commercially available as BM-1000®, BM-1100®, or the like of BM Chemie Inc.; F 142D®, F 172®, F 173®, F 183®, or the like of Dainippon Ink Kagaku Kogyo Co., Ltd.; FULORAD FC-135®, FC-170C®, FC-430®, FC-431®, or the like of Sumitomo 3M Co., Ltd.; SURFLON S-112®, S-113®, S-131®, S-141®, S-145®, or the like of ASAHI Glass Co., Ltd.; SH-28PA®, SH-190®, SH-193®, SZ-6032®, SF-8428®, or the like.

The surfactant may be included in an amount of, e.g., about 0.001 parts by weight to about 5 parts by weight, based on 100 parts by weight of the photosensitive resin composition. If the surfactant is included within the range, the coating uniformity may be secured, stains are not found, and the wetting property to the glass substrate may be improved.

In an implementation, the photosensitive resin composition may further include other additives, e.g., an oxidization inhibitor, a stabilizer, or the like in a predetermined amount, unless properties are deteriorated.

According to some example embodiments, a photosensitive resin layer manufactured using the photosensitive resin composition according to some example embodiments may be provided.

According to some example embodiments, a color filter including the photosensitive resin layer may be provided.

A pattern-forming process in the color filter may be as follows.

The process may include coating the photosensitive resin composition according to some example embodiments on a support substrate in a method of spin coating, slit coating, inkjet printing, or the like; drying the coated positive photosensitive resin composition to form a photosensitive resin composition film; exposing the positive photosensitive resin composition film to light; developing the exposed positive photosensitive resin composition film in an alkali aqueous solution to obtain a photosensitive resin layer; and heat-treating the photosensitive resin layer.

Some example embodiments may provide a CMOS image sensor or OLEDos (on silicon) device including the color filter.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

### (Synthesis of Compounds)

### Synthesis Example 1: Synthesis of Compound Represented by Chemical Formula A

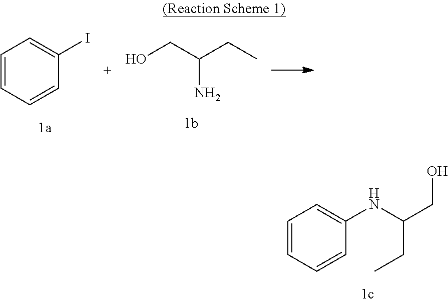

Compound 1a (12 mmol) and Compound 1b (10 mmol) with CuCl (0.5 mmol) and NaOH (20 mmol) were dissolved in 2-propanol and then, reacted for 12 hours by heating to 90° C. After cooling to ambient temperature, distilled water was added thereto and then, extracted with DCM (dichloromethane). An organic layer therefrom was passed through MgSO₄, concentrated under a reduced pressure, and treated through column chromatography (eluent: n-Hex/EtOAc) to obtain Compound 1c.

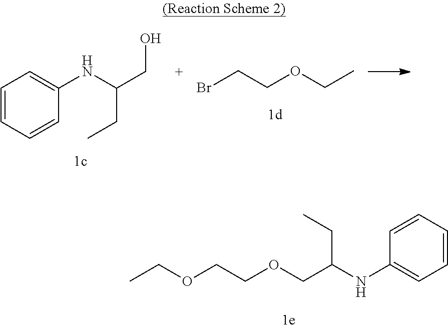

NaH (60% in mineral oil, 15 mmol) was added dropwise to a solution of Compound 1c (10 mmol) in THF (tetrahydrofuran) at 0° C. for 30 minutes. After adding Compound 1d (10.5 mmol), the reaction temperature was raised to ambient temperature and the reaction was conducted overnight. The aqueous layer was extracted using diethylether. The organic layer was passed through MgSO₄ and concentrated under reduced pressure, and then Compound 1e was obtained through column chromatography (eluent: n-Hex/EtOAc).

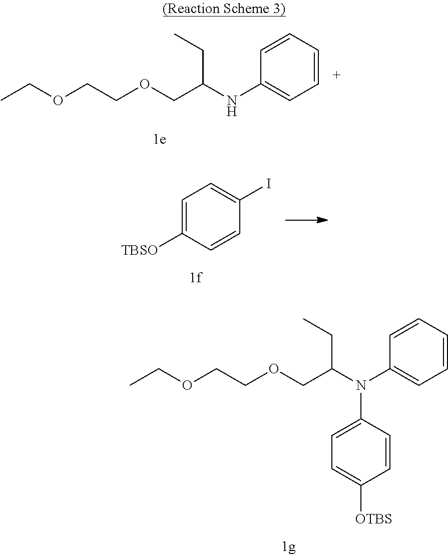 [SMILES: CCOCCOCC(CC)Nc1ccccc1 ; CC(C)(C)[Si](C)(C)Oc1ccc(I)cc1 ; CCOCCOCC(CC)N(c1ccccc1)c1ccc(O[Si](C)(C)C(C)(C)C)cc1]

Compound 1e (10 mmol) and Compound 1f (11 mmol) with K₂CO₃ (15 mmol) and Pd(PPh₃)₄ (1.0 mmol) were added to a mixed solution of 1,4-dioxane/H₂O (v/v=4/1) and then, reacted under reflux. After cooling to ambient temperature, an organic layer was separated therefrom. The organic layer was passed through MgSO₄, concentrated under a reduced pressure, and treated through column chromatography (eluent: n-Hex/EtOAc) to obtain Compound 1g.

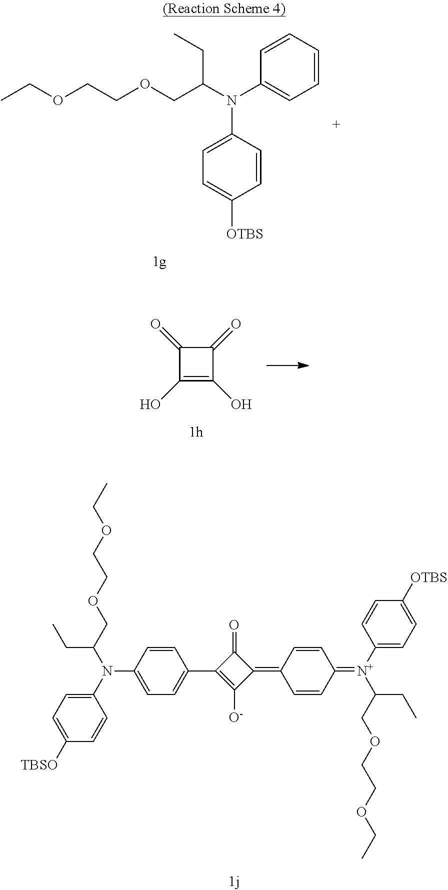 [SMILES: CCOCCOCC(CC)N(c1ccccc1)c1ccc(O[Si](C)(C)C(C)(C)C)cc1 ; O=c1c(O)c(O)c1=O ; CCOCCOCC(CC)N(c1ccc(O[Si](C)(C)C(C)(C)C)cc1)c1ccc(C2=C([O-])C(=C3C=CC(=[N+](c4ccc(O[Si](C)(C)C(C)(C)C)cc4)C(CC)COCCOCC)C=C3)C2=O)cc1]

Compound 1g (10 mmol) and Compound 1h (7 mmol) were dissolved in n-butanol at ambient temperature, and triethylorthoformate (30 mmol) was added thereto and then, reacted for 4 hours by heating to 90° C. After removing the solvent under a reduced pressure, n-hexane was added to the remaining reaction mixture and then, stirred at 0° C. for 15 minutes. A solid obtained therefrom by filtration under a reduced pressure was washed and dried, obtaining Compound 1i.

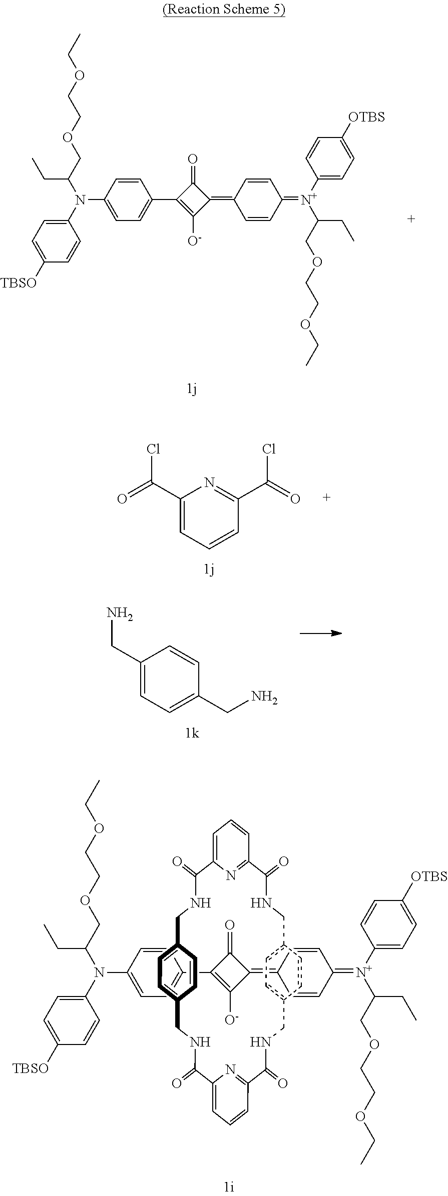

After dissolving Compound 1i (10 mmol) in chloroform and then, cooling the solution to 0° C., two other solutions were prepared: (Solution A) Compound 1k (30 mmol) and triethylamine (60 mmol) were dissolved in chloroform, and (Solution B) Compound 1j (30 mmol) was dissolved in chloroform. These two solutions, in the order of Solution A then Solution B, were slowly added to the solution of Compound 1i and then, stirred at ambient temperature for 2 hours. After removing the solvent under a reduced pressure, the residue was redissolved in EtOAC to precipitate polymer byproducts. After removing the byproducts by filtration under a reduced pressure, the solvent was removed under a reduced pressure. After dissolving the residue in chloroform, the above process was twice repeated. A material corresponding to Compound 1l was obtained by purification through column chromatography (eluent: DCM-EtOAc).

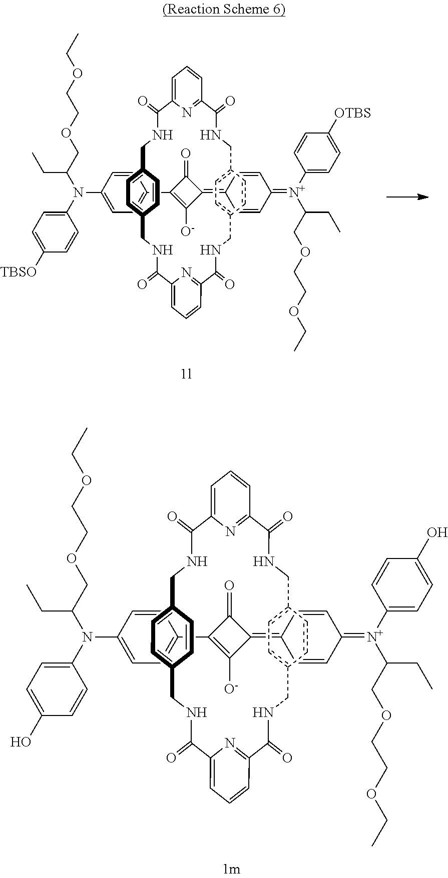

Compound 1l (10 mmol) was dissolved in THF to obtain a solution, to which 1 M TBAF (tetrabutylammonium fluoride, 22 mmol) was added and then, stirred at ambient temperature for 2 hours. After checking if Compound 1l was completely consumed through thin-layer chromatography, the solvent was removed under a reduced pressure. Acetone/H₂O (v/v=1/1) was added thereto and then, stirred at 0° C. for 30 minutes, and a solid produced therein was separated by filtration under a reduced pressure.

The solid was washed with distilled water and dried at 40° C. in a vacuum oven to obtain Compound 1m.

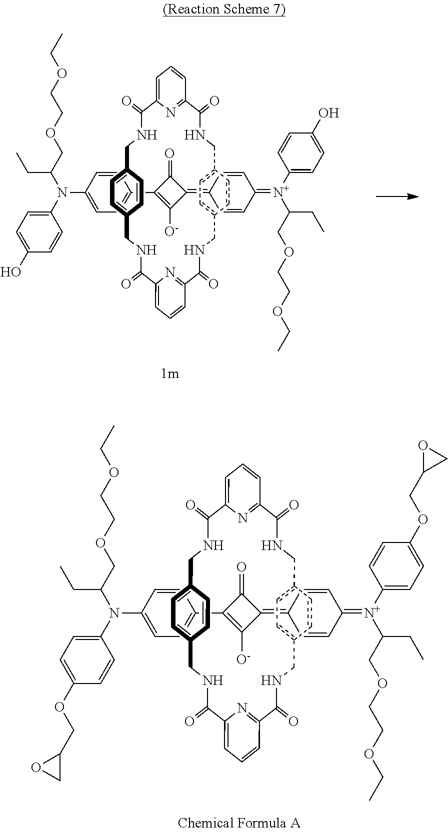

Compound 1m (10 mmol) was dissolved in DMSO (dimethylsulfoxide) to obtain a solution, to which KOH (50 mmol) and ECH (epichlorohydrin, 100 mmol) were added and then, stirred at 60° C. for 2 hours. Subsequently, distilled water was added thereto and then, extracted by using DCM. An organic layer therefrom was washed with brine and then, dried and concentrated. A material corresponding to Chemical Formula A was obtained by purification through Column chromatography (eluent: DCM-EtOAc).

The HRMS analysis results of Chemical Formula A are as follows.

m/z calcd for C₈₀H₈₇N₈O₁₄ ([M+H]⁺), 1383.6336; found, 1383.6336.

### Synthesis Example 2: Synthesis of Compound Represented by Chemical Formula B

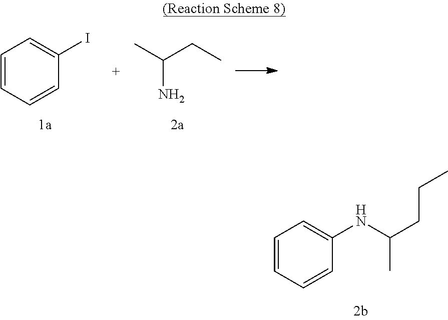

Compound 2b was synthesized in the same manner as the synthesis of Compound 1c, except that Compound 2a was used instead of Compound 1b.

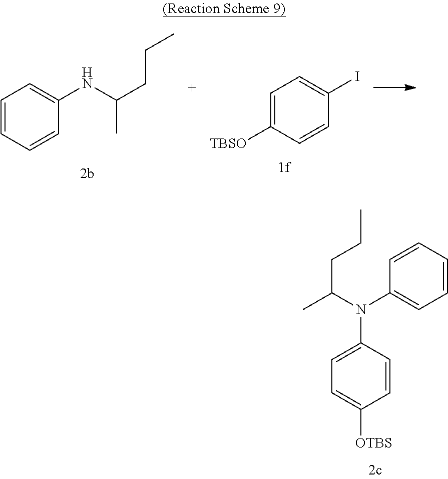 [SMILES: CCCC(C)Nc1ccccc1 ; CC(C)(C)[Si](C)(C)Oc1ccc(I)cc1 ; CCCC(C)N(c1ccccc1)c1ccc(O[Si](C)(C)C(C)(C)C)cc1]

Compound 2c was synthesized in the same manner as the synthesis of Compound 1g, except that Compound 2b was used instead of Compound 1e.

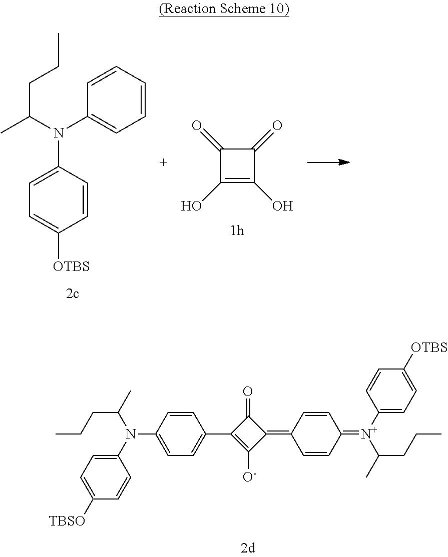 [SMILES: CCCC(C)N(c1ccccc1)c1ccc(O[Si](C)(C)C(C)(C)C)cc1 ; O=c1c(O)c(O)c1=O ; CCCC(C)N(c1ccc(O[Si](C)(C)C(C)(C)C)cc1)c1ccc(C2=C([O-])C(=C3C=CC(=[N+](c4ccc(O[Si](C)(C)C(C)(C)C)cc4)C(C)CCC)C=C3)C2=O)cc1]

Compound 2d was synthesized in the same manner as the method of synthesizing Compound 1i except that Compound 2c was used instead of Compound 1g.

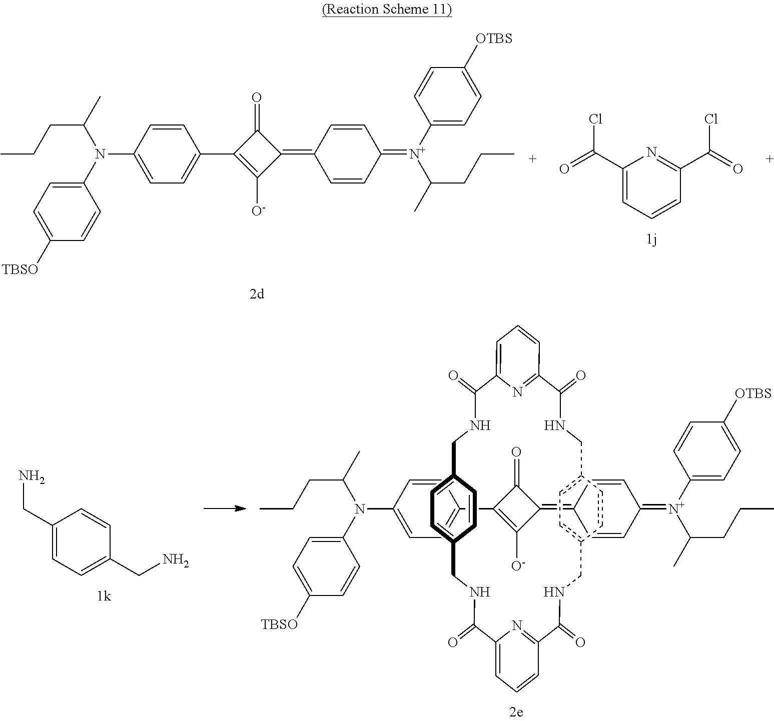

Compound 2e was synthesized in the same manner as the method of synthesizing Compound 1l except that Compound 2d was used instead of Compound 1i.

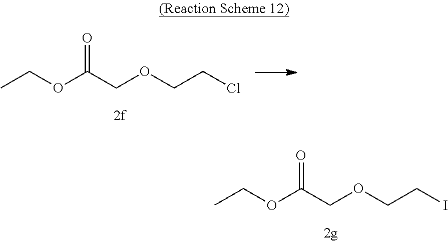

NaI (50 mmol) was added to a solution of Compound 2f (10 mmol) in acetone, and the mixture was stirred overnight at 50° C. The solvent was removed under reduced pressure, distilled water was added, and extraction was performed using DCM. The organic layer was washed with brine, dried and concentrated to obtain Compound 2g.

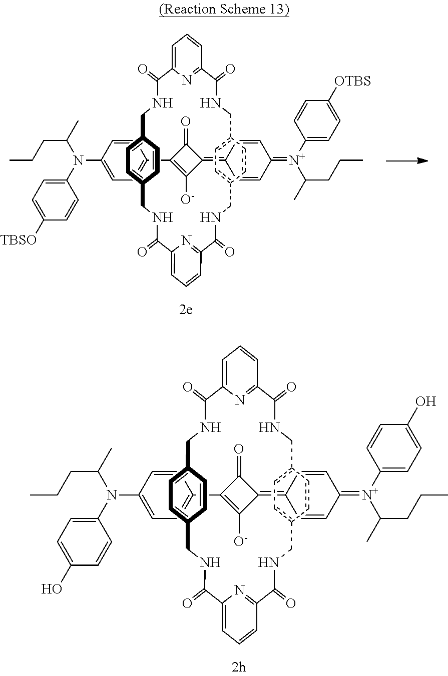

Compound 2h was synthesized in the same manner as the synthesis of Compound 1m, except that Compound 2e was used instead of Compound 1l.

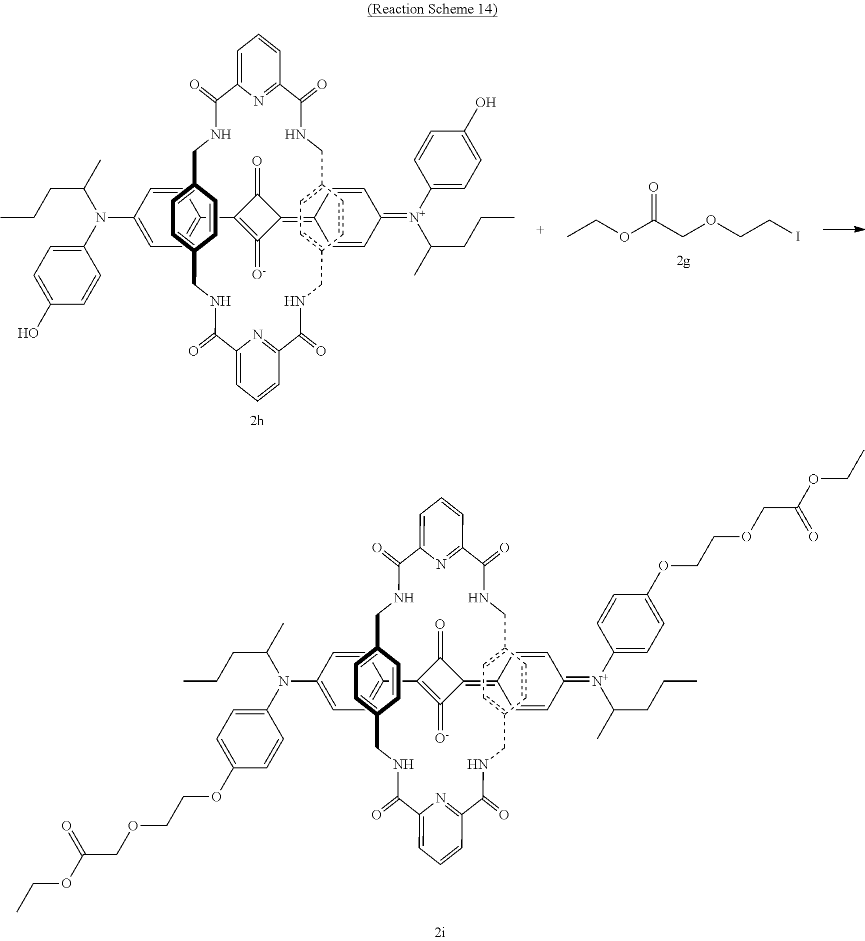

To a solution of Compound 2h (4.36 mmol) and K₂CO₃ (43.6 mmol) dissolved in DMSO, Compound 2g (43.6 mmol) was added and the temperature was raised to 80° C. After stirring overnight, the temperature was slowly cooled to ambient temperature. Diethylether was added to the reaction mixture to produce a solid, which was separated through reduced pressure filtration. The solid was washed with a 10% aqueous sodium chloride solution and dried overnight in a vacuum oven at 40° C. to obtain Compound 2i.

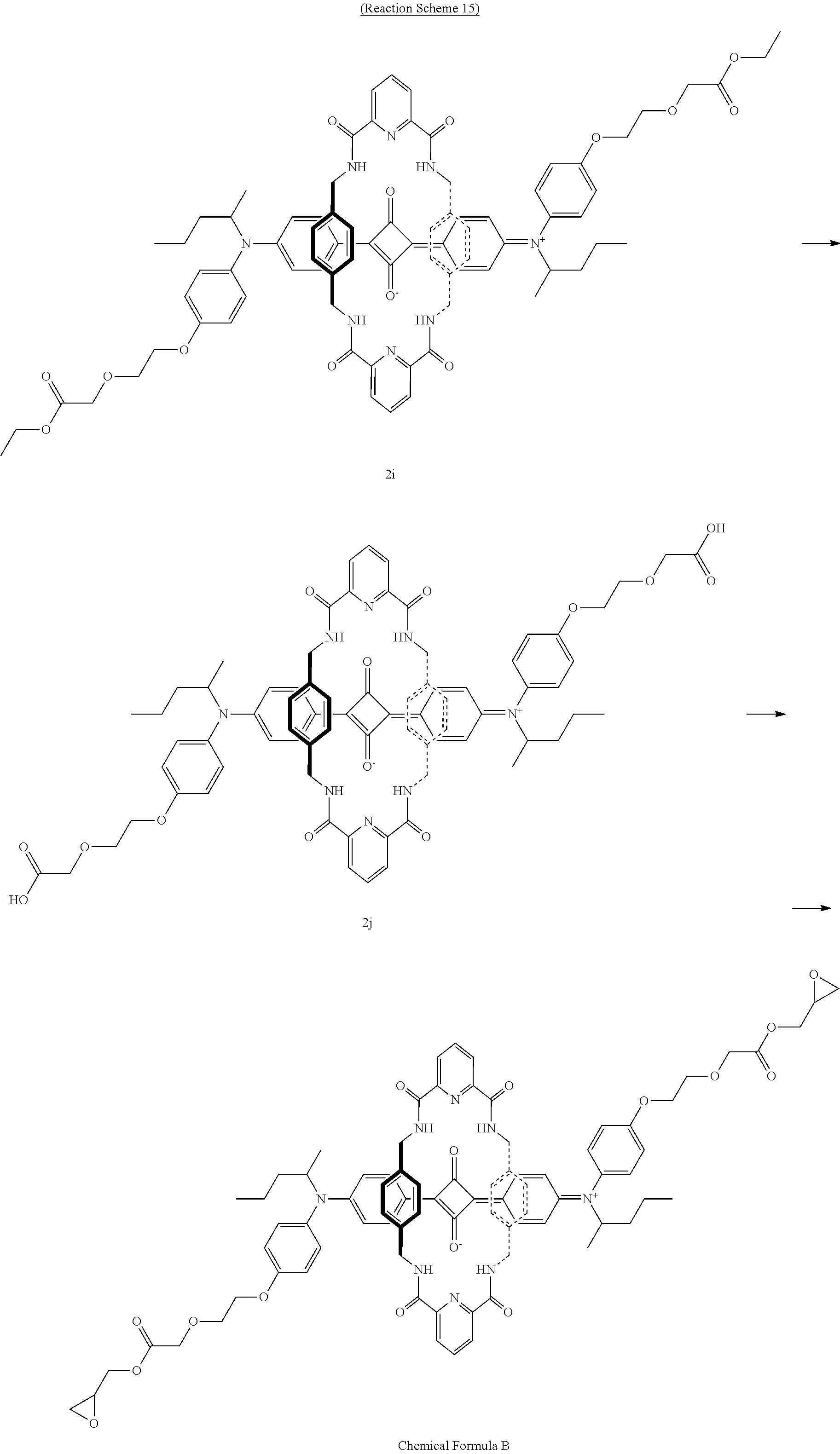

A 1 N NaOH aqueous solution (20 mmol) was added to a solution of Compound 2i (2.1 mmol) in THF, and the mixture was stirred overnight at ambient temperature. 1 N HCl aqueous solution was slowly added to the reaction mixture, and then extracted using DCM. The organic layer was passed through MgSO₄ and the solvent was removed under reduced pressure to obtain Compound 2j, which was used in the next reaction without further purification. KOH (5.0 mmol) and ECH (epichlorohydrin, 10 mmol) were added to a solution of Compound 2j (1.0 mmol) in DMSO, and stirred at 60° C. for 2 hours. After adding distilled water, extraction was performed using DCM. The organic layer was washed with brine, then dried and concentrated. The material corresponding to Chemical Formula B was obtained by purification through column chromatography (eluent: DCM-EtOAc).

The HRMS analysis results of Chemical Formula B are as follows.

m/z calcd for C₈₂H₈₇N₈O₁₆ ([M+H]⁺), 1439.6235; found, 1439.6236.

### Synthesis Example 3: Synthesis of Compound Represented by Chemical Formula C

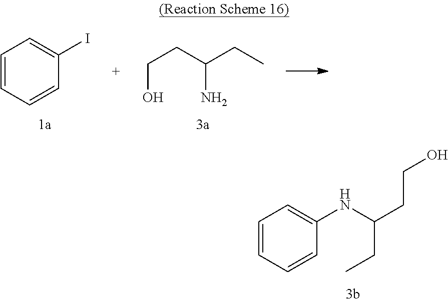

Compound 3b was synthesized in the same manner as the method of synthesizing Compound 1c except that Compound 3a was used instead of Compound 1b.

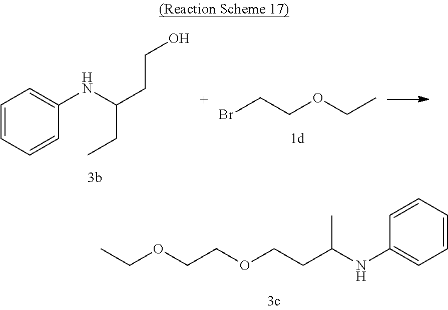

Compound 3c was synthesized in the same manner as the synthesis of Compound 1e, except that Compound 3b was used instead of Compound 1c.

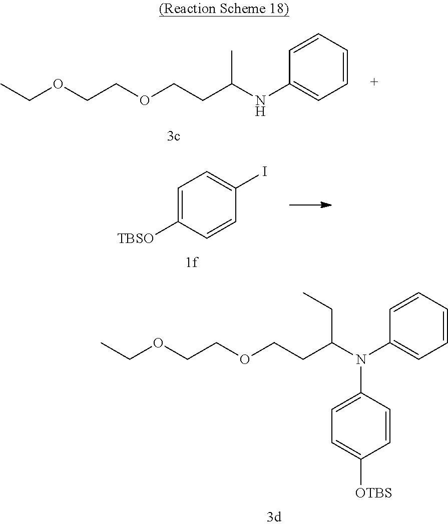 [SMILES: CCOCCOCCC(C)Nc1ccccc1 ; CC(C)(C)[Si](C)(C)Oc1ccc(I)cc1 ; CCOCCOCCC(CC)N(c1ccccc1)c1ccc(O[Si](C)(C)C(C)(C)C)cc1]

Compound 3d was synthesized in the same manner as the synthesis of Compound 1g, except that Compound 3c was used instead of Compound 1e.

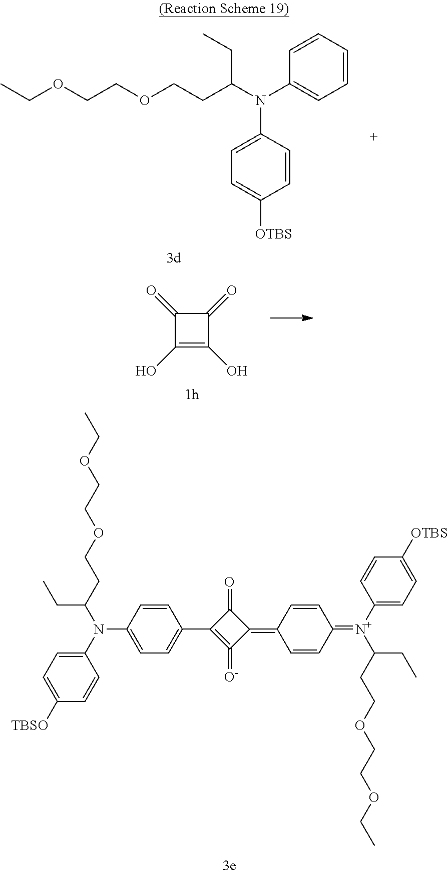

Compound 3e was synthesized in the same manner as the synthesis of Compound 1i, except that Compound 3d was used instead of Compound 1g.

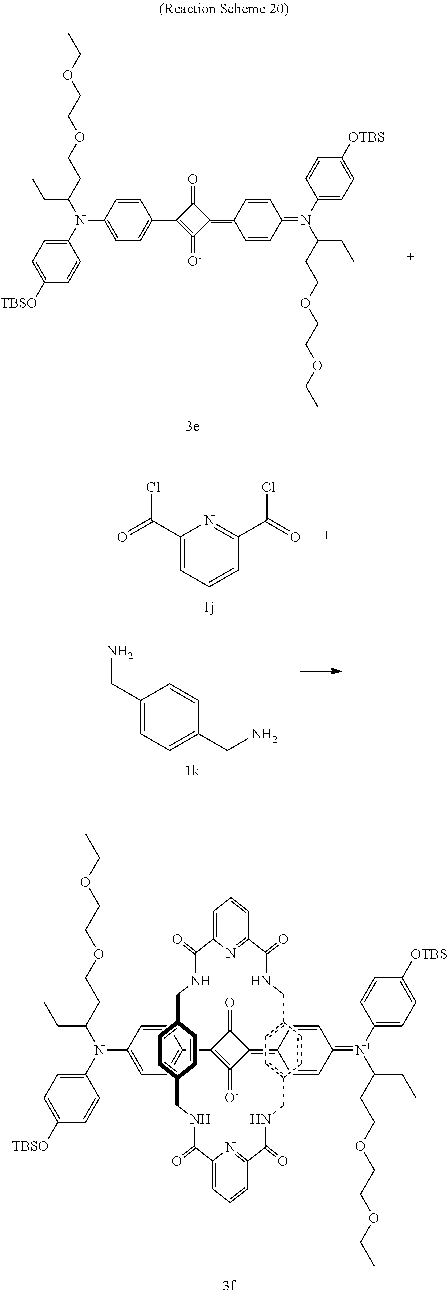

Compound 3f was synthesized in the same manner as the synthesis of Compound 1l, except that Compound 3e was used instead of Compound 1i.

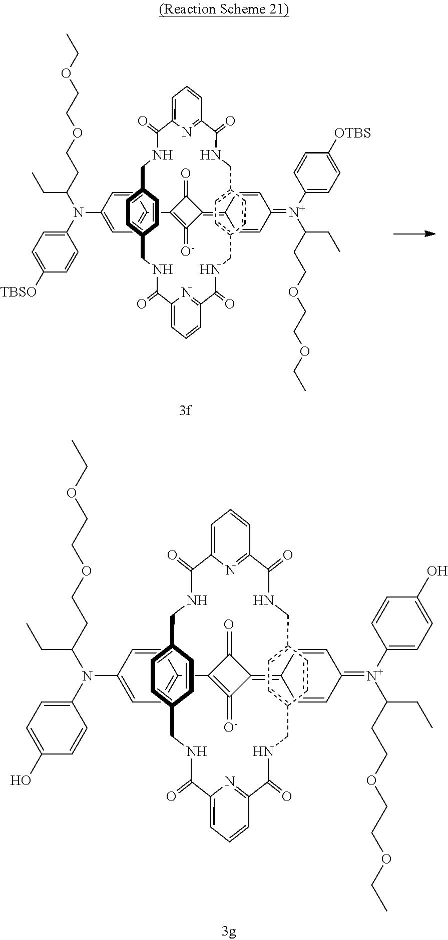

Compound 3g was synthesized in the same manner as the synthesis of Compound 1m, except that Compound 3f was used instead of Compound 1l.

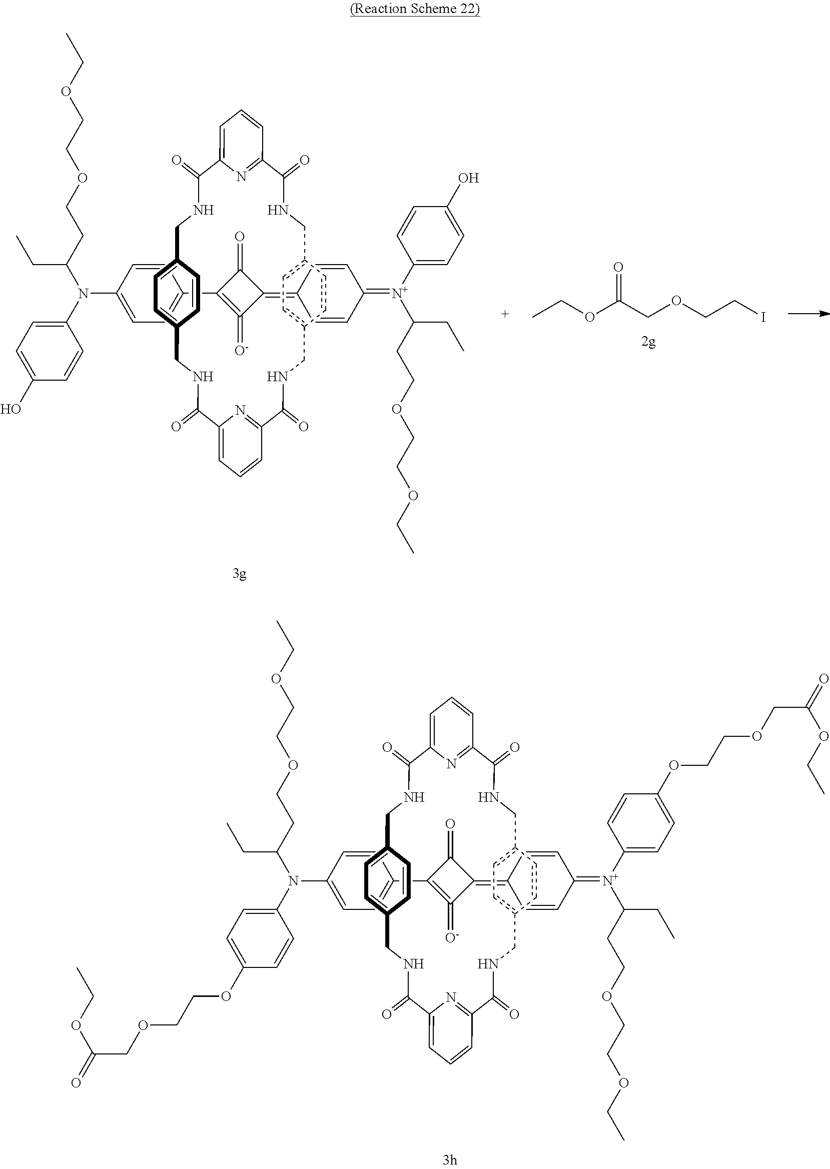

Compound 3h was synthesized in the same manner as the synthesis of Compound 2i, except that Compound 3g was used instead of Compound 2h.

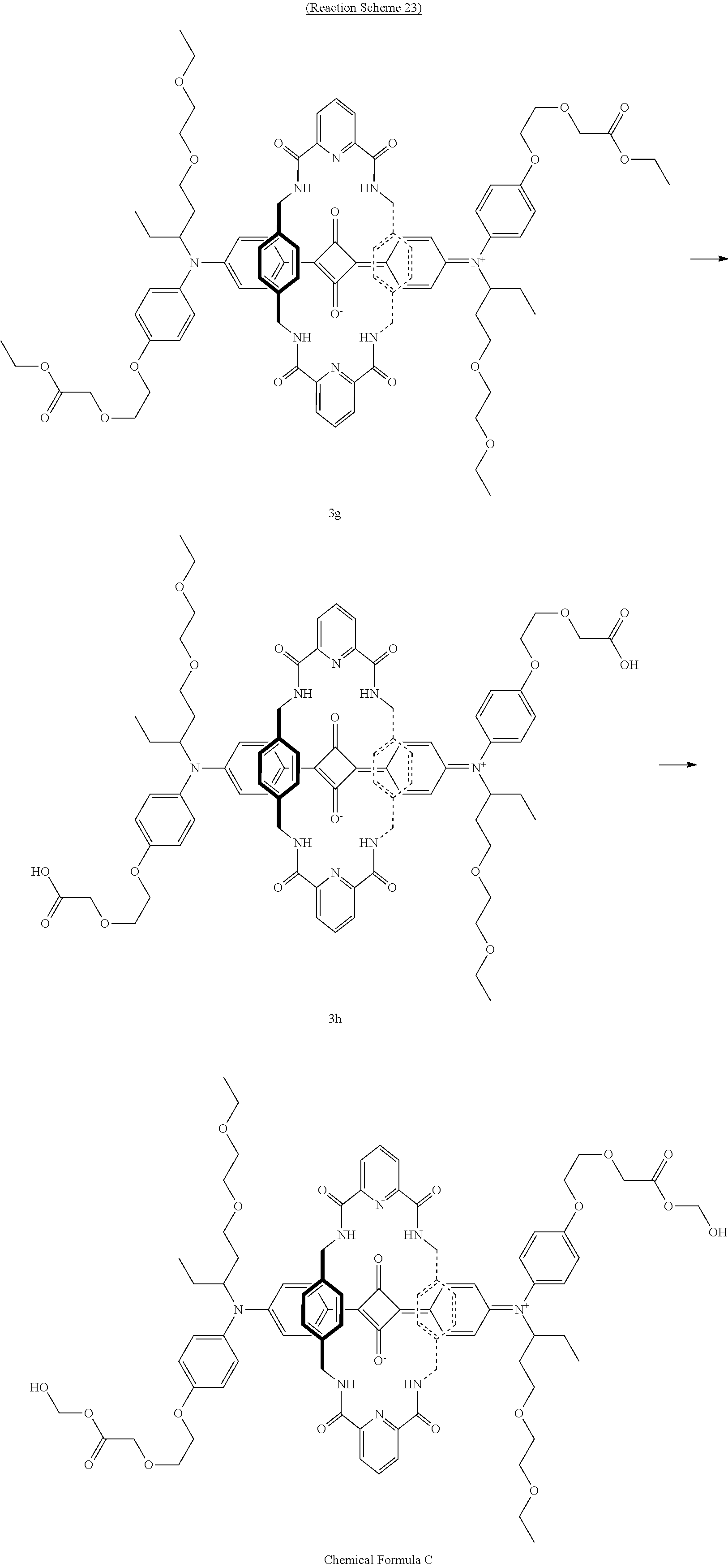

A material corresponding to Chemical Formula C was synthesized in the same manner as the synthesis of a material corresponding to Chemical Formula B, except that the Compound 3h was used instead of Compound 2i.

The HRMS analysis results of Chemical Formula C are as follows.

m/z calcd for C₉₀H₁₀₃N₈O₂₀ ([M+H]⁺), 1615.7283; found, 1615.7283.

### Comparative Synthesis Example 1: Synthesis of Compound Represented by Chemical Formula D

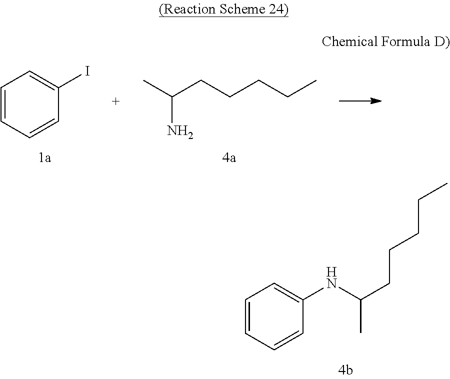

Compound 4b was synthesized in the same manner as the synthesis of Compound 1c, except that Compound 4a was used instead of Compound 1b.

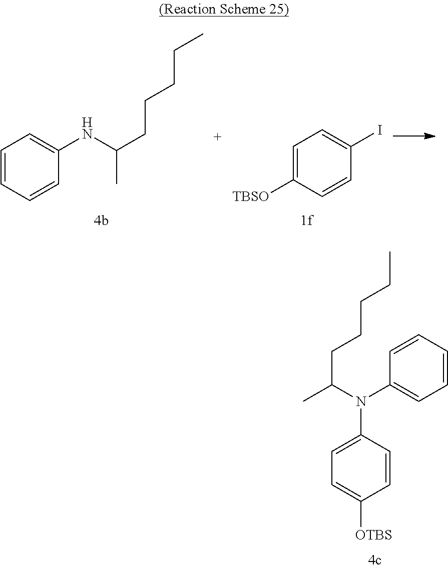 [SMILES: CCCCCC(C)Nc1ccccc1 ; CC(C)(C)[Si](C)(C)Oc1ccc(I)cc1 ; CCCCCC(C)N(c1ccccc1)c1ccc(O[Si](C)(C)C(C)(C)C)cc1]

Compound 4c was synthesized in the same manner as the synthesis of Compound 1g, except that Compound 4b was used instead of Compound 1e.

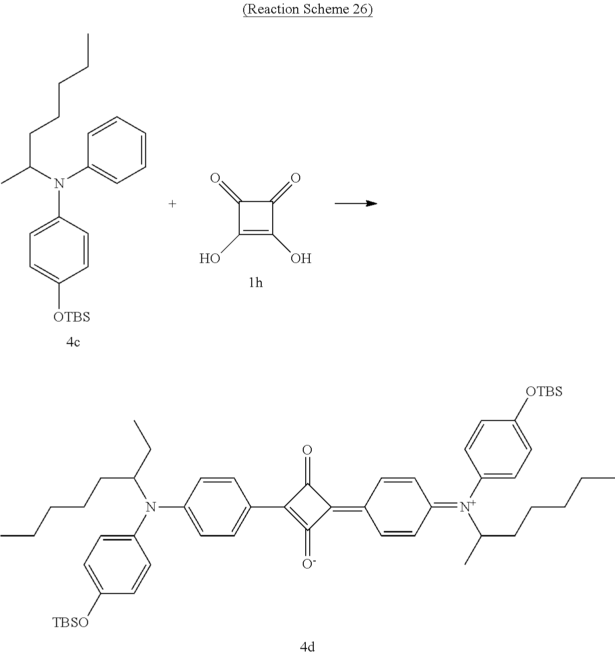

Compound 4d was synthesized in the same manner as the synthesis of Compound 1i, except that Compound 4c was used instead of Compound 1g.

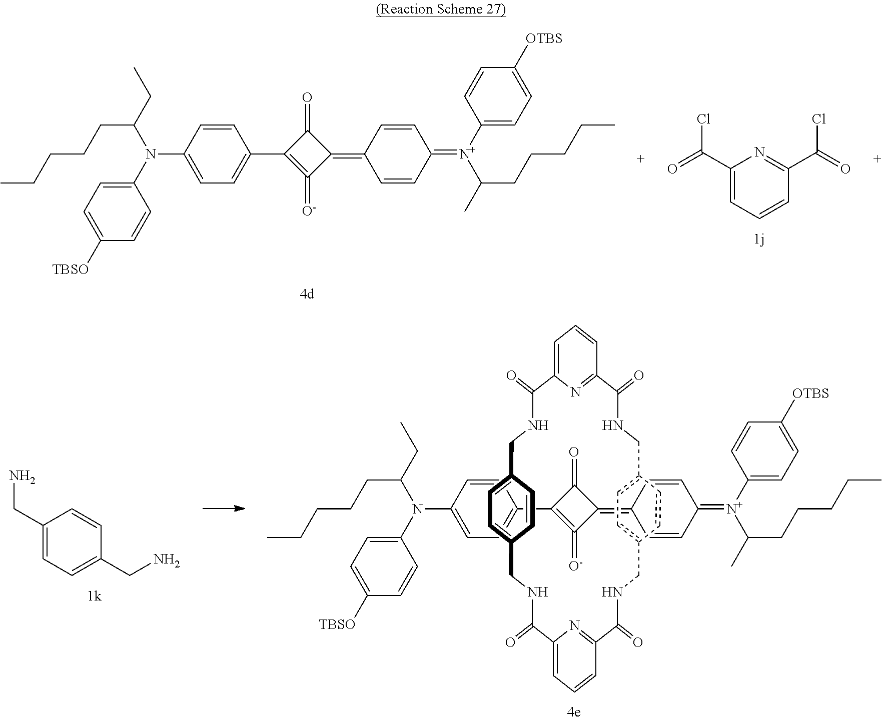

Compound 4e was synthesized in the same manner as the synthesis of Compound 1l, except that Compound 4d was used instead of Compound 1i.

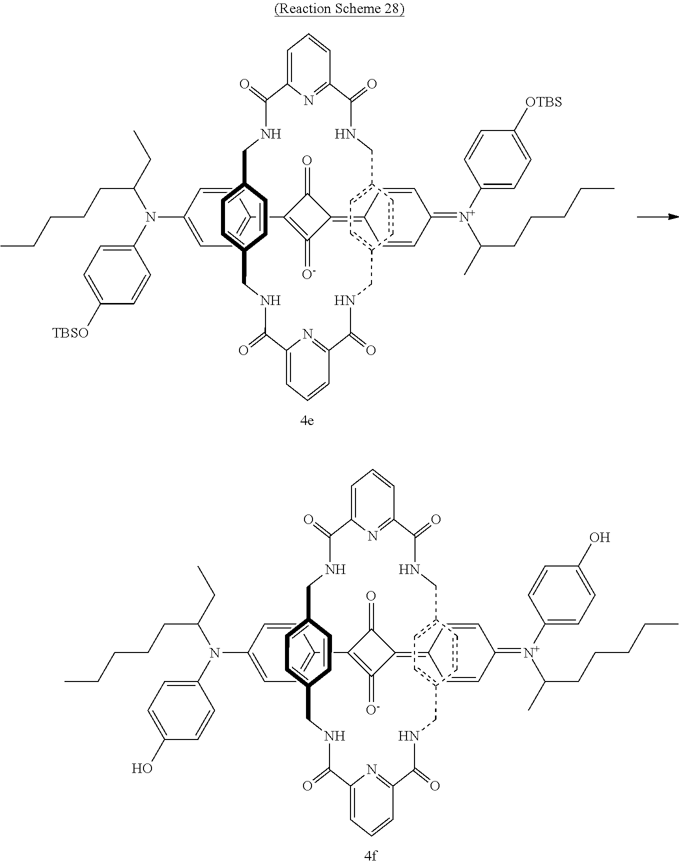

Compound 4f was synthesized in the same manner as the synthesis of Compound 1m, except that Compound 4e was used instead of Compound 1l.

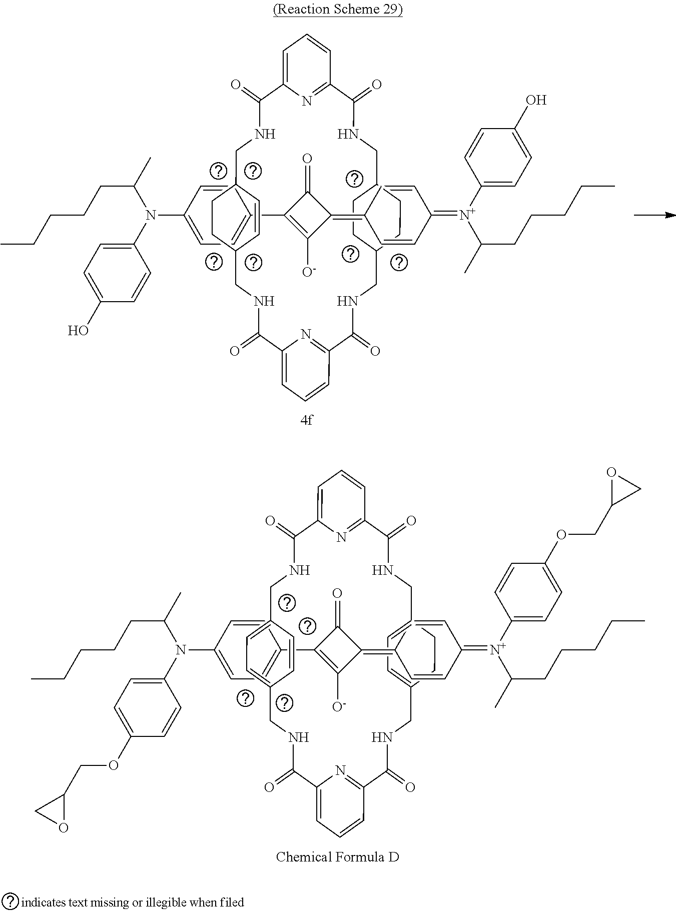

A material corresponding to Chemical Formula D was synthesized in the same manner as the synthesis of a substance corresponding to Chemical Formula A, except that Compound 4f was used instead of Compound 1m.

The HRMS analysis results of Chemical Formula D are as follows.

m/z calcd for C₇₈H₈₃N₈O₁₀ ([M+H]⁺), 1291.6227; found, 1291.6228.

### Comparative Synthesis Example 2: Synthesis of Compound Represented by Chemical Formula E

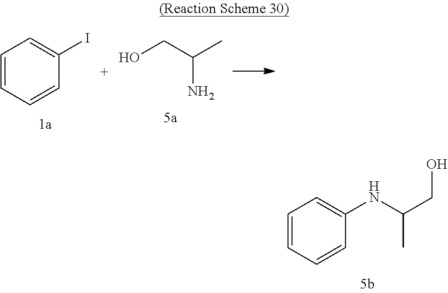

Compound 5b was synthesized in the same manner as the synthesis of Compound 1c, except that Compound 5a was used instead of Compound 1b.

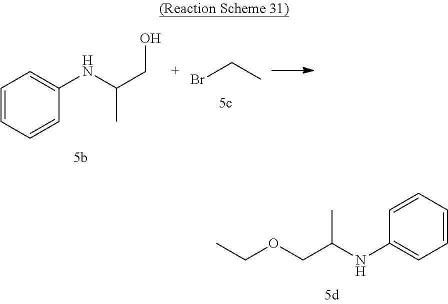

Compound 5d was synthesized in the same manner as the synthesis of Compound 1e, except that Compound 5b was used instead of Compound 1c, and Compound 5c was used instead of Compound 1d.

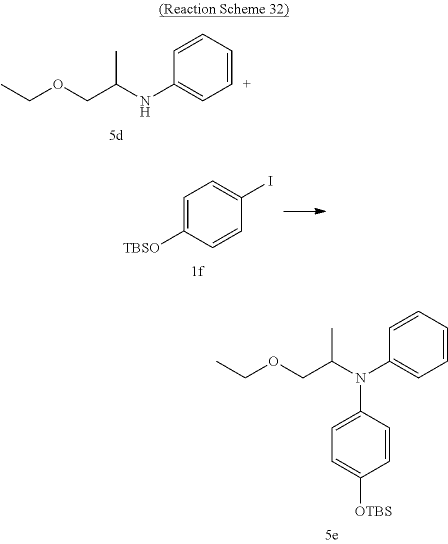 [SMILES: CCOCC(C)NC1=C[C+]=CC=C1 ; CC(C)(C)[Si](C)(C)Oc1ccc(I)cc1 ; CCOCC(C)N(c1ccccc1)c1ccc(O[Si](C)(C)C(C)(C)C)cc1]

Compound 5e was synthesized in the same manner as the synthesis of Compound 1g, except that Compound 5d was used instead of Compound 1e.

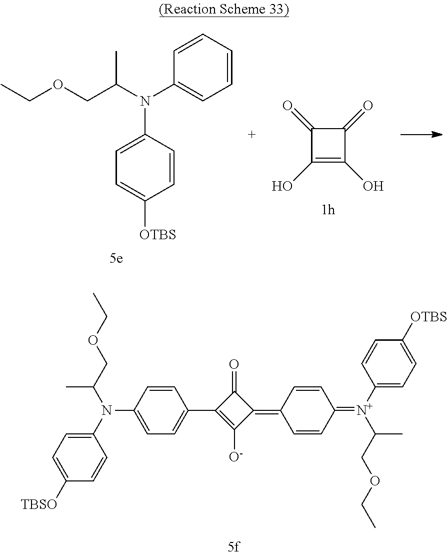 [SMILES: CCOCC(C)N(c1ccccc1)c1ccc(O[Si](C)(C)C(C)(C)C)cc1 ; O=c1c(O)c(O)c1=O ; CCOCC(C)N(c1ccc(O[Si](C)(C)C(C)(C)C)cc1)c1ccc(C2=C([O-])C(=C3C=CC(=[N+](c4ccc(O[Si](C)(C)C(C)(C)C)cc4)C(C)COCC)C=C3)C2=O)cc1]

Compound 5f was synthesized in the same manner as the synthesis of Compound 1i, except that Compound 5e was used instead of Compound 1g.

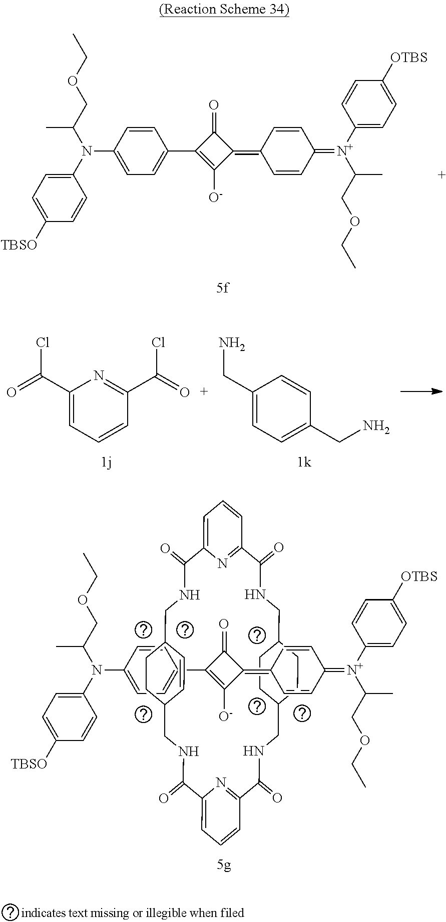

Compound 5g was synthesized in the same manner as the synthesis of Compound 1l, except that Compound 5f was used instead of Compound 1i.

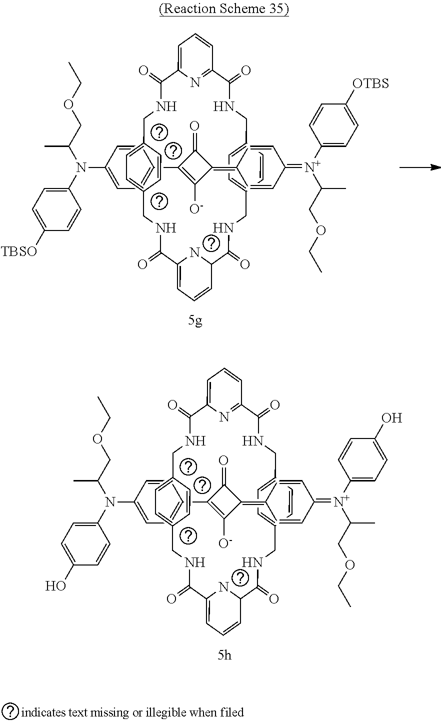

Compound 5h was synthesized in the same manner as the synthesis of Compound 1m, except that Compound 5g was used instead of Compound 1l.

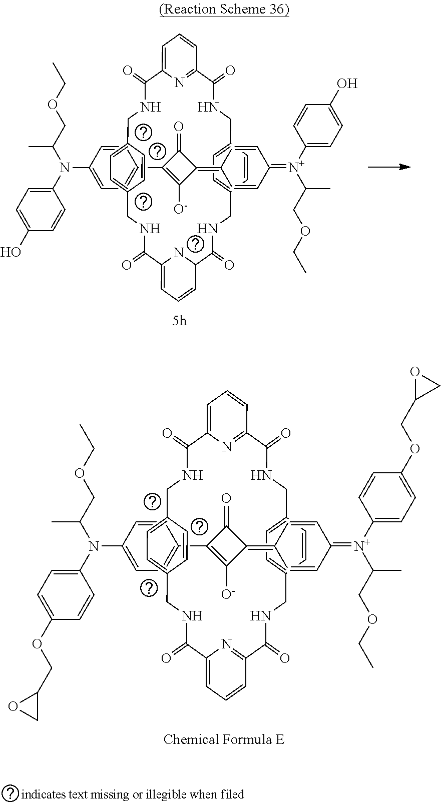

A material corresponding to Chemical Formula E was synthesized in the same manner as the synthesis of the material corresponding to Chemical Formula A, except that Compound 5h was used instead of Compound 1m.

The HRMS analysis results of Chemical Formula E are as follows.

m/z calcd for C74H75N₈O₁₂ ([M+H]⁺), 1267.5499; found, 1267.5499.

### Comparative Synthesis Example 3: Synthesis of Compound Represented by Chemical Formula F

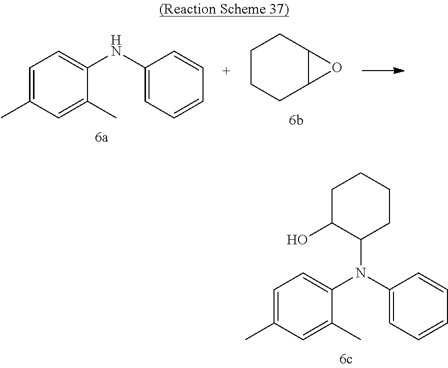 [SMILES: Cc1ccc(Nc2ccccc2)c(C)c1 ; C1CCC2OC2C1 ; Cc1ccc(N(c2ccccc2)C2CCCCC2O)c(C)c1]

NaH (60% in mineral oil, 15 mmol) was added dropwise to a solution of Compound 6a (10 mmol) in DMF (N,N-dimethylformamide) at ambient temperature for 30 minutes. After adding Compound 6b (10.5 mmol), the reaction temperature was raised to 90° C. and the reaction was performed overnight. The aqueous layer was extracted using DCM. The organic layer was passed through MgSO₄ and concentrated under reduced pressure, and then Compound 6c was obtained through column chromatography (eluent: n-Hex/EtOAc).

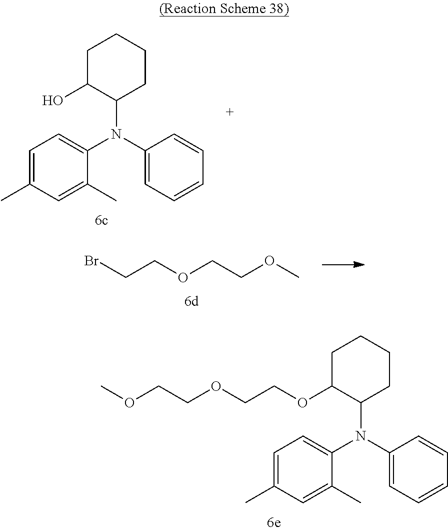

Compound 6e was synthesized in the same manner as the synthesis of Compound 1e, except that Compound 6c was used instead of Compound 1c and Compound 6d was used instead of Compound 1d.

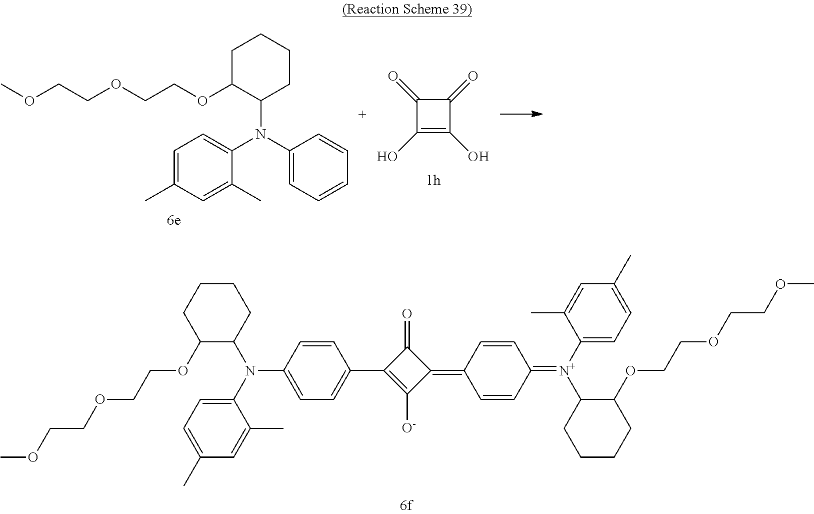

Compound 6f was synthesized in the same manner as the synthesis of Compound 1i, except that Compound 6e was used instead of Compound 1g.

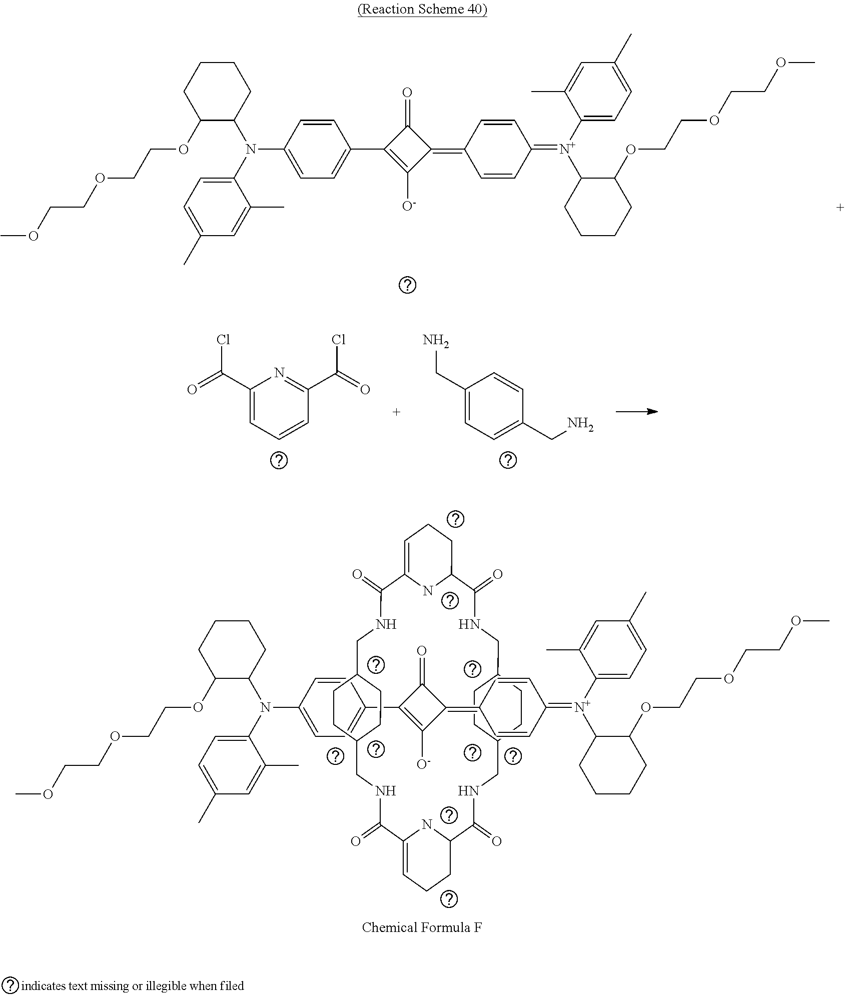

A material corresponding to Chemical Formula F was synthesized in the same manner as the synthesis of Compound 1l, except that Compound 6f was used instead of Compound 1i.

The HRMS analysis results of Chemical Formula F are as follows.

m/z calcd for C₈₄H₉₅N₈O₁₂ ([M+H]⁺), 1407.7064; found, 1407.7065.

### Comparative Synthesis Example 4: Synthesis of Compound Represented by Chemical Formula G

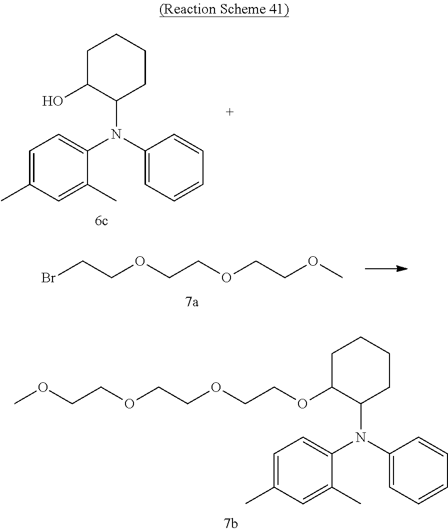

Compound 7b was synthesized in the same manner as the synthesis of Compound 1e, except that Compound 6c was used instead of Compound 1c and Compound 7a was used instead of Compound 1d.

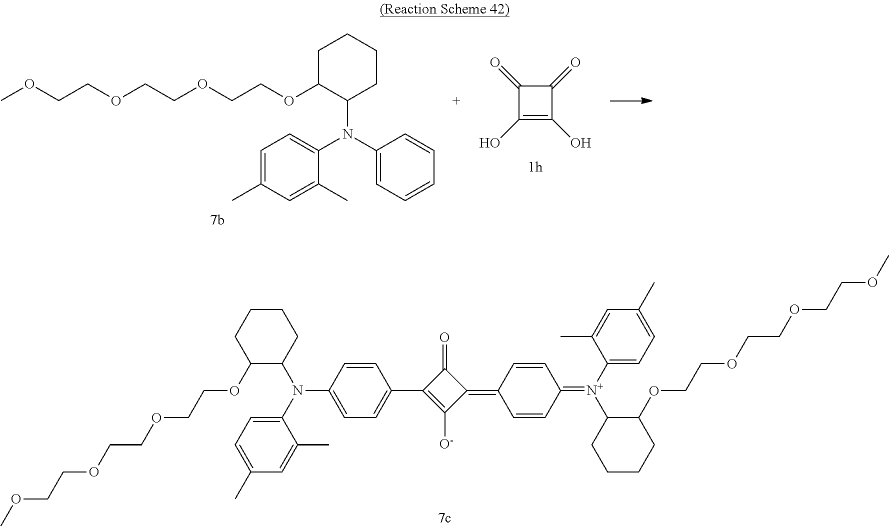

Compound 7c was synthesized in the same manner as the synthesis of Compound 1i, except that Compound 7b was used instead of Compound 1g.

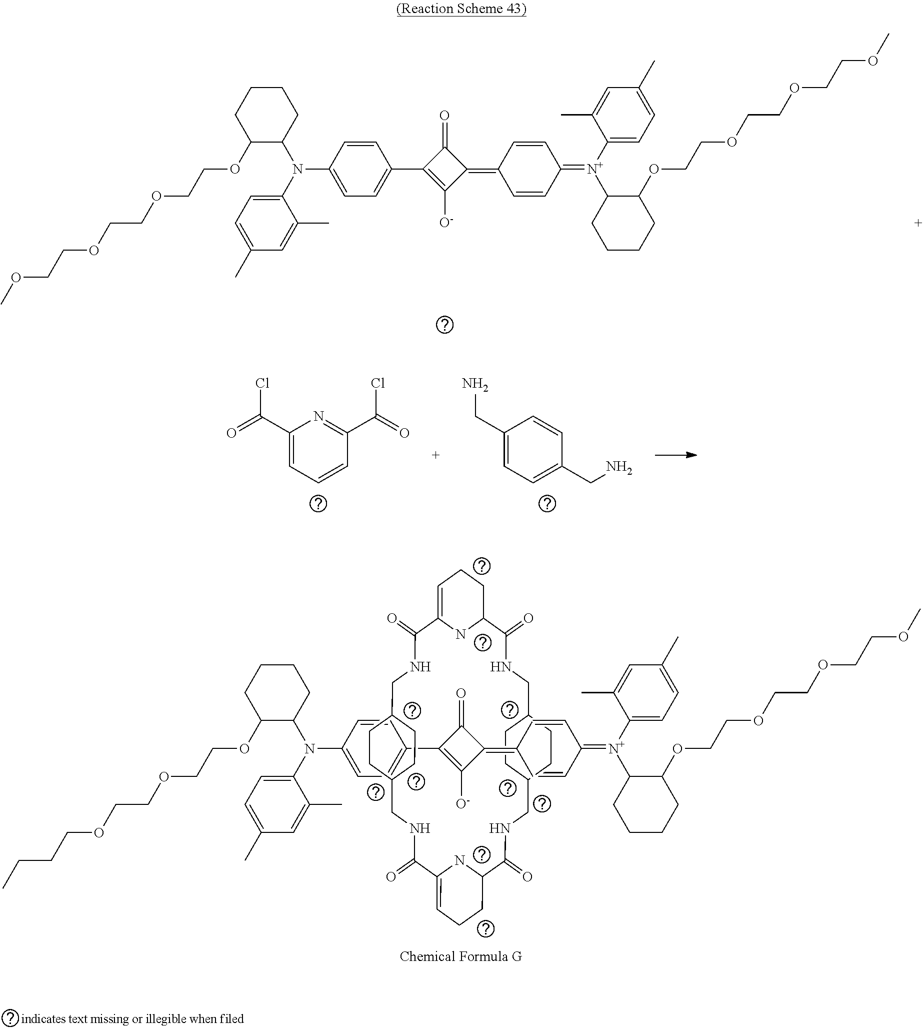

A material corresponding to Chemical Formula G was synthesized in the same manner as the synthesis of Compound 1l, except that Compound 7c was used instead of Compound 1i.

The HRMS analysis results of Chemical Formula G are as follows.

m/z calcd for C₈₈H₁₀₃N₈O₁₄ ([M+H]⁺), 1495.7588; found, 1495.7588.

### (Synthesis of Photosensitive Resin Compositions)

### Example 1

The following components were mixed in each composition shown in Table 1 to prepare a photosensitive resin composition according to Example 1.

Specifically, a photopolymerization initiator was dissolved in a solvent, the solution was stirred at ambient temperature for 2 hours, a binder resin and a photopolymerizable monomer were added thereto, and the obtained mixture was stirred at ambient temperature for 2 hours. Subsequently, the compound (represented by Chemical Formula A) according to Synthesis Example 1 was added thereto as a colorant and then, stirred for 1 hour at ambient temperature. Then, the product was filtered three times to remove impurities and prepare a photosensitive resin composition.

TABLE 1
(unit: wt %)

| Raw materials | | | Amount |
|---|---|---|---|
| Colorant | Dye | Compound of Synthesis Example 1 | 20 |

| | | |
|---|---|---|
| Binder resin | (A)/(B) = 15/85 (w/w), | 3.5 |
| | molecular weight (Mw) = 22,000 g/mol | |
| | (A): methacrylic acid | |
| | (B): benzylmethacrylate | |
| Photopolymerizable | Dipentaerythritolhexaacrylate (DPHA) | 8.0 |
| monomer | | |
| Photopolymerization | 1,2-octandione | 1.0 |
| initiator | 2-dimethylamino-2-(4-methyl-benzyl)-1- | 0.5 |
| | (4-morpholin-4-yl-phenyl)-butan-1-one | |
| Solvent | Cyclohexanone | 37.0 |
| | PGMEA (Propylene Glycol Monomethyl | 30.0 |
| | Ether Acetate) | |

| | |
|---|---|
| Total | 100.00 |

### Example 2

A photosensitive resin composition was prepared according to the same method as Example 1 except that the compound (represented by Chemical Formula B) according to Synthesis Example 2 was used instead of the compound (represented by Chemical Formula A) according to Synthesis Example 1.

### Example 3

A photosensitive resin composition was prepared according to the same method as Example 1 except that the compound (represented by Chemical Formula C) according to Synthesis Example 3 was used instead of the compound (represented by Chemical Formula A) according to Synthesis Example 1.

### Comparative Example 1

A photosensitive resin composition was prepared according to the same method as Example 1 except that the compound (represented by Chemical Formula D) according to Comparative Synthesis Example 1 was used instead of the compound (represented by Chemical Formula A) according to Synthesis Example 1.

### Comparative Example 2

A photosensitive resin composition was prepared according to the same method as Example 1 except that the compound (represented by Chemical Formula E) according to Comparative Synthesis Example 2 was used instead of the compound (represented by Chemical Formula A) according to Synthesis Example 1.

### Comparative Example 3

A photosensitive resin composition was prepared according to the same method as Example 1 except that the compound (represented by Chemical Formula F) according to Comparative Synthesis Example 3 was used instead of the compound (represented by Chemical Formula A) according to Synthesis Example 1.

### Comparative Example 4

A photosensitive resin composition was prepared according to the same method as Example 1 except that the compound (represented by Chemical Formula G) according to Comparative Synthesis Example 4 was used instead of the compound (represented by Chemical Formula A) according to Synthesis Example 1.

### Evaluation: Measurement of Developability of Compositions

The photosensitive resin compositions prepared in Examples 1 to 3 and Comparative Examples 1 to 5 were spin-coated on a silicon wafer to a thickness of 0.6 μm and then prebaked on a hot plate at 100° C. for 3 minutes. The formed thin film was exposed at 200 mJ/cm² using a KrF scanner exposure device (248 nm). Then, the unexposed region was removed with 0.2% TMAH (tetramethylammonium hydroxide) in the developer (SVS, SSP-200) to form a colored pattern. Then, it was post-baked on a 230° C. hot plate for 5 minutes.

The developability of the obtained coloring pattern was evaluated by checking whether the pattern was formed and the surrounding residue using Hitachi's CD-SEM (scanning electron microscopy), and the results are shown in Table 2.

TABLE 2

| | Developability |
|---|---|
| Example 1 | ⊚ |
| Example 2 | ◯ |
| Example 3 | ⊚ |
| Comparative Example 1 | X |
| Comparative Example 2 | X |
| Comparative Example 3 | Δ |
| Comparative Example 4 | Δ |

(Criteria)
⊚: No residue;
◯: Slight residue only around the pattern periphery;
Δ: Residue occurs over the entire area;
X: Undeveloped.

From the Table 2, the photosensitive resin compositions of Examples 1 to 3 including a high content of a core-shell compound according to the above embodiments have excellent developability, making them highly suitable for use in CMOS image sensors or OLED devices.

By way of summation and review, the CMOS image sensor is developed along a technology trend of increasing the number of pixels and decreasing a size for realizing high-definition and down-sizing a device. As the pixels become smaller, there may be a limit to manufacture a fine pattern by using a pigment, and accordingly, a dye for compensating for this may be considered. However, the dye may have issues in terms of processability during the pattern manufacture, compared with the pigment. For example, the dye may have issues in terms of chemical resistance, because the pigment is fine particles and has crystallinity and thus insufficient solubility and thereby is not eluted in a solvent such as PGMEA after the baking, but the dye is an amorphous solid and thus dissolved out in the solvent after the baking. The CMOS image sensor may use a high content of a colorant and thus use a binder resin or a monomer at a relatively lower ratio, and the chemical resistance of the dye may be difficult to improve. Some example embodiments provide a core-shell compound constituting a green pixel in a color filter for a CMOS image sensor.

Some example embodiments may provide a photosensitive resin composition including the compound.

Some example embodiments may provide a photosensitive resin layer manufactured using the photosensitive resin composition.

Some example embodiments may provide a color filter including the photosensitive resin layer.

Some example embodiments may provide a CMOS image sensor including the color filter.

The core-shell compound according to some example embodiments may have excellent developability in itself and may maintain excellent developability even after curing and heat processes, and a photosensitive resin composition including the same as a dye may form a fine pattern and provide a green color filter for a CMOS image sensor.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A core-shell compound, comprising a core represented by Chemical Formula 1 and a shell surrounding the core, the shell being represented by Chemical Formula 2: 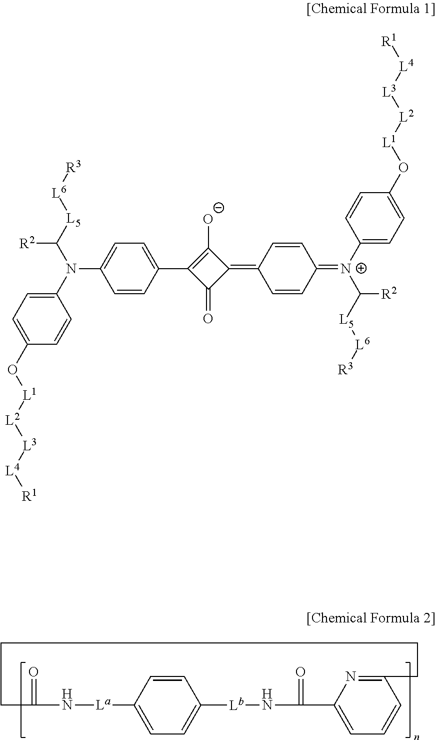
wherein, in Chemical Formula 1 and Chemical Formula 2,
R¹ is a thermosetting group,
R² and R³ are each independently a hydrogen atom, a substituted or unsubstituted C1 to C20 alkyl group, or a substituted or unsubstituted C1 to C20 alkoxy group,
L¹ and L⁶ are each independently a substituted or unsubstituted C1 to C10 alkylene group or a group represented by Chemical Formula L, provided that at least one of L¹ and L⁶ is a group represented by Chemical Formula L,
L², L⁴, Lₐ, and L_{b} are each independently a single bond or a substituted or unsubstituted C1 to C10 alkylene group,
L³ is a single bond or an ester group represented by *—C(═O)O—* or *—OC(═O)—*,
L⁵ is a single bond or a substituted or unsubstituted C1 to C10 alkylene group, and
n is an integer greater than or equal to 2, 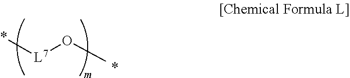
wherein, in Chemical Formula L,
L⁷ is a substituted or unsubstituted C1 to C10 alkylene group, and
m is an integer of 2 to 10.

2. The core-shell compound as claimed in claim 1, wherein the thermosetting group includes a substituted or unsubstituted epoxy group, a substituted or unsubstituted oxetane group, or a combination thereof.

3. The core-shell compound as claimed in claim 1, wherein:
R¹ is a thermosetting group,
R² and R³ are each independently a hydrogen atom or a substituted or unsubstituted C1 to C20 alkyl group,
L¹ and L⁵ are each independently a substituted or unsubstituted C1 to C10 alkylene group,
L² to L⁴ are each independently a single bond, and
L⁶ is a group represented by Chemical Formula L.

4. The core-shell compound as claimed in claim 1, wherein:
R¹ is a thermosetting group,
R² and R³ are each independently a hydrogen atom or a substituted or unsubstituted C1 to C20 alkyl group, L¹ is a group represented by Chemical Formula L,
L², L⁴, and L⁶ are each independently a substituted or unsubstituted C1 to C10 alkylene group,
L³ is an ester group represented by *—C(═O)O—* or *—OC(═O)—*, and
L⁵ is a single bond or a substituted or unsubstituted C1 to C10 alkylene group.

5. The core-shell compound as claimed in claim 1, wherein:
R¹ is a thermosetting group,
R² and R³ are each independently a hydrogen atom or a substituted or unsubstituted C1 to C20 alkyl group,
L¹ and L⁶ are each independently a group represented by Chemical Formula L,
L² and L⁴ are each independently a substituted or unsubstituted C1 to C10 alkylene group,
L³ is an ester group represented by *—C(═O)O—* or *—OC(═O)—*, and
L⁵ is a substituted or unsubstituted C1 to C10 alkylene group.

6. The core-shell compound as claimed in claim 1, wherein the core represented by Chemical Formula 1 has a maximum absorption wavelength at about 610 nm to about 640 nm.

7. The core-shell compound as claimed in claim 1, wherein the shell represented by Chemical Formula 2 is represented by Chemical Formula 2-1:

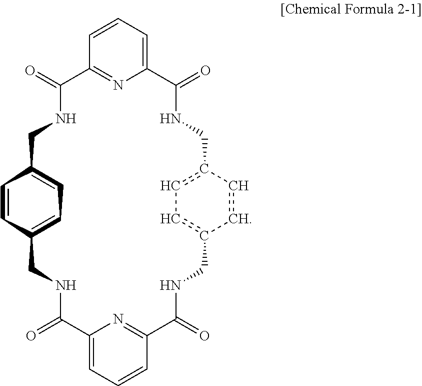

8. The core-shell compound as claimed in claim 1, wherein the core-shell compound is represented by one of Chemical Formula A to Chemical Formula C:

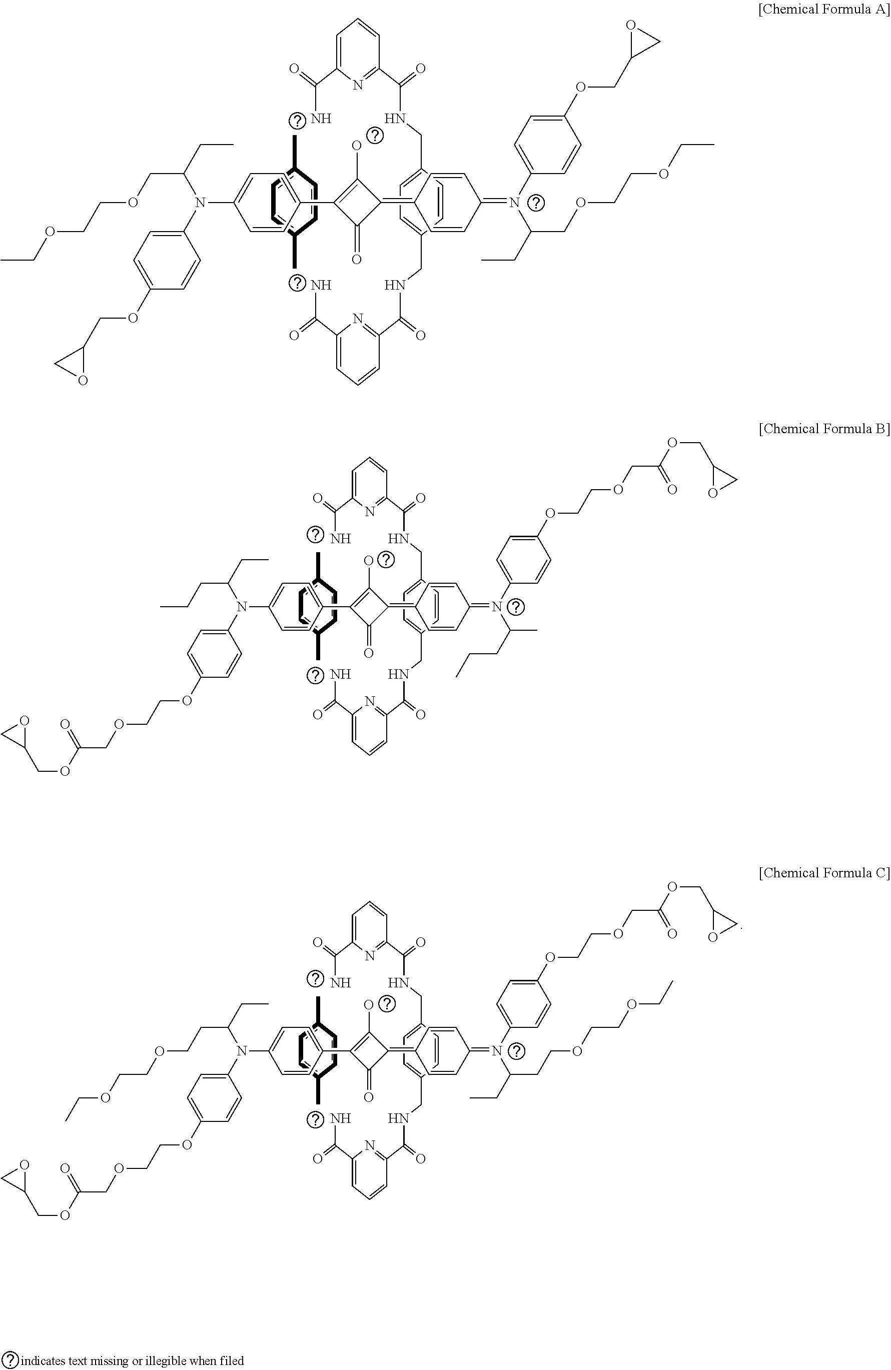

9. The core-shell compound as claimed in claim 1, wherein the core-shell compound is a green dye.

10. A photosensitive resin composition comprising the core-shell compound as claimed in claim 1.

11. The photosensitive resin composition as claimed in claim 10, further including a binder resin, a photopolymerizable monomer, a photopolymerization initiator, a pigment, or a solvent.

12. The photosensitive resin composition as claimed in claim 10, wherein the photosensitive resin composition is used for a CMOS image sensor.

13. A photosensitive resin layer manufactured using the photosensitive resin composition as claimed in claim 10.

14. A color filter comprising the photosensitive resin layer as claimed in claim 13.

15. A CMOS image sensor comprising the color filter as claimed in claim 14.
